(19) United States
(12) Patent Application Publication
(10) Pub. No.: US 2015/0158956 A1
(43) Pub. Date: Jun. 11, 2015

(54) MULTIVALENT ANTIVIRAL COMPOSITIONS, METHODS OF MAKING, AND USES THEREOF

(71) Applicants: The Charles Stark Draper Laboratory, Inc., Cambridge, MA (US); The Massachusetts Institute of Technology, Cambridge, MA (US); University of California, Santa Barbara (UCSB), Santa Barbara, CA (US); University of Massachusetts Medical School, Boston, MA (US)

(72) Inventors: James Comolli, Boxborough, MA (US); Robert Finberg, Newtonville, MA (US); Deborah K. Fygenson, Santa Barbara, CA (US); Zachary Shriver, Winchester, MA (US); Ram Sasisekharan, Cambridge, MA (US); Jennifer Wang, Shrewsbury, MA (US); Karthik Viswanathan, Waltham, MA (US)

(21) Appl. No.: 14/519,009

(22) Filed: Oct. 20, 2014

Related U.S. Application Data
(60) Provisional application No. 61/892,834, filed on Oct. 18, 2013.

(51) Int. Cl. C08B 37/00; A61K 31/727; A61K 47/48

(52) U.S. Cl. CPC C08B 37/0078; A61K 47/48053; A61K 31/727

(57) ABSTRACT

This disclosure provides compositions, kits, and methods useful for treating or preventing viral infection. The methods comprise administering to a subject an effective amount of a sulfated polysaccharide composition.

### CROSS-REFERENCE TO RELATED APPLICATION

This application claims priority to U.S. Provisional Application No. 61/892,834 filed Oct. 18, 2013, the content of which is incorporated herein by reference in its entirety.

### GOVERNMENT SUPPORT

This invention was made with government support under contract number W911NF-10-1-0268 awarded by the Defense Advanced Research Projects Agency, Defense Sciences Office. The U.S. Government has certain rights in the invention.

### BACKGROUND

The following description is provided to assist the understanding of the reader. None of the information provided or references cited is admitted to be prior art to the present technology.

Respiratory syncytial virus (RSV) is a major cause of respiratory illness in youths and the immunocompromised. There are currently only two FDA approved treatments for RSV, palivizumab and ribavirin. Palivizumab is a mono-clonal antibody that is only partially effective. Ribavirin is a small molecule inhibitor with significant and toxic side effects. Both are expensive treatments.

RSV is known to bind to heparin sulfate located on the cell membrane. The binding of RSV to the heparin sulfate is believed to facilitate viral entry into the cell.

### SUMMARY

The present technology relates generally to sulfated polysaccharides and sulfated polysaccharide compositions, which can prevent or treat viral infection.

In one aspect, the present technology provides a sulfated polysaccharide having the formula of GlcUA(2S)-GlcNS(6S)-Ido(2S)-GlcNS(6S)-Ido(2S)-GlcNS(6S)-Ido(2S)-GlcNS(6S).

In one aspect, the present technology provides for a polysaccharide composition including at least one glycolipid, wherein the glycolipid includes at least one sulfated polysaccharide linked to a lipid and wherein the sulfated polysaccharide includes between 3 to 10 monosaccharide units. In some implementations, the polysaccharide composition further includes a mixture of lipids, wherein the mixture of lipids and the glycolipid are in the form of a liposome, wherein the sulfated polysaccharide is displayed on a surface of the liposome.

In some implementations, the sulfated polysaccharide includes between 1-4 sulfur atoms per disaccharide. In some implementations, the sulfated polysaccharide includes an octasaccharide, wherein the octasaccharide includes three sulfur atoms per disaccharide. In some implementations, the sulfur atoms are in the form of a sulfate.

In some implementations, the sulfated polysaccharide includes at least one monosaccharide select from the group consisting of Ido(2S), Iduronate, 2-O sulfo-iduronate, and 2,3-split uronic acid. In some implementations, the sulfated polysaccharide is GlcUA(2S)-GlcNS(6S)-Ido(2S)-GlcNS(6S)-Ido(2S)-GlcNS(6S)-Ido(2S)-GlcNS(6S).

In some implementations, polysaccharide composition inhibits a viral infection. In some implementations, the viral infection is selected from the group consisting of respiratory syncytial virus (RSV), influenza virus, herpes simplex viruses 1 and 2, cytomegalovirus (CMV), vaccinia virus, vesicular stomatitis virus (VSV), Sindbis virus, HIV, human papillomavirus (HPV), influenza A virus, and alphaviruses. In some implementations, the composition binds to the F and G envelop glycoproteins of the virus.

In one aspect, the present technology provides for a polysaccharide composition including at least one sulfated polysaccharide and a carrier molecule or a support substrate, wherein the sulfated polysaccharide is displayed on a surface of the carrier molecule or the support substrate and wherein the sulfated polysaccharide includes between 3 to 10 monosaccharide units.

In some implementations, the polysaccharide composition also includes at least one glycolipid linked to the surface of the carrier molecule or the support substrate, wherein the glycolipid includes at least one sulfated polysaccharide linked to a lipid. In some implementations, the carrier molecule or the support substrate is selected from the group consisting of peptides, polypeptide, protein, carbohydrate, nanoparticles (e.g., metal, polymeric), polymers (e.g., polymer beads), glass beads, magnetic particles, nucleic acid, small molecule, cells, virus, dendrimer, particle, bead, macromolecule, and solid lipid particle.

In some implementations, the sulfated polysaccharide includes between 1-4 sulfur atoms per disaccharide. In some implementations, the sulfated polysaccharide includes an octasaccharide, wherein the octasaccharide includes three sulfur atoms per disaccharide. In some implementations, the sulfur atoms are in the form of a sulfate.

In some implementations, the sulfated polysaccharide includes at least one monosaccharide select from the group consisting of Ido(2S), Iduronate, 2-O sulfo-iduronate, and 2,3-split uronic acid. In some implementations, the sulfated polysaccharide is GlcUA(2S)-GlcNS(6S)-Ido(2S)-GlcNS(6S)-Ido(2S)-GlcNS(6S)-Ido(2S)-GlcNS(6S).

In some implementations, the composition inhibits a viral infection. In some implementations, the viral infection is selected from the group consisting of respiratory syncytial virus (RSV), influenza virus, herpes simplex viruses 1 and 2, cytomegalovirus (CMV), vaccinia virus, vesicular stomatitis virus (VSV), Sindbis virus, HIV, human papillomavirus (HPV), influenza A virus, and alphaviruses. In some implementations, the composition binds to the F and G envelop glycoproteins.

In one aspect, the present technology provides for a method of treating or preventing a viral infection including administering a therapeutic amount of a sulfated polysaccharide, wherein the sulfated polysaccharide includes between 3 to 10 monosaccharide units.

In some implementations, the sulfated polysaccharide of the method includes between 1-4 sulfur atoms per disaccharide. In some implementations, the sulfated polysaccharide of the method includes an octasaccharide, wherein the octasaccharide includes three sulfur atoms per disaccharide. In some implementations, the sulfur atoms are in the form of a sulfate.

In some implementations, the sulfated polysaccharide of the method includes at least one monosaccharide select from the group consisting of Ido(2S), Iduronate, 2-O sulfo-iduronate, and 2,3-split uronic acid. In some implementations, the sulfated polysaccharide of the method is GlcUA(2S)-GlcNS(6S)-Ido(2S)-GlcNS(6S)-Ido(2S)-GlcNS(6S)-Ido(2S)-GlcNS(6S).

In some implementations, the sulfated polysaccharide of the method is linked to a surface of a carrier molecule or support substrate.

In one aspect, the present technology provides for a method of treating or preventing a viral infection including administering a therapeutic amount of a polysaccharide composition, the composition including at least one glycolipid, wherein the glycolipid includes at least one sulfated polysaccharide linked to a lipid and wherein the sulfated polysaccharide includes between 3 to 10 monosaccharide units.

In some implementations, the polysaccharide composition of the method also includes a mixture of lipids, wherein the mixture of lipids and at least one glycolipid are in the form of a liposome, wherein the sulfated polysaccharide is displayed on a surface of the liposome.

In some implementations, the sulfated polysaccharide composition of the method also includes a carrier molecule or support substrate, wherein at least one glycolipid is linked to the surface of the carrier molecule or the support substrate. In some implementations, the carrier molecule or the support substrate is selected from the group consisting of peptides, polypeptide, protein, carbohydrate, nanoparticles (e.g., metal, polymeric), polymers (e.g., polymer beads), glass beads, magnetic particles, nucleic acid, small molecule, cells, virus, dendrimer, particle, bead, macromolecule, and solid lipid particle.

In some implementations, the sulfated polysaccharide of the method includes between 1-4 sulfur atoms per disaccharide. In some implementations, the sulfated polysaccharide of the method includes an octasaccharide, wherein the octasaccharide includes three sulfur atoms per disaccharide. In some implementations, the sulfur atoms are in the form of a sulfate.

In some implementations, the sulfated polysaccharide of the method includes at least one monosaccharide select from the group consisting of Ido(2S), Iduronate, 2-O sulfo-iduronate, and 2,3-split uronic acid. In some implementations, the sulfated polysaccharide of the method is GlcUA(2S)-GlcNS(6S)-Ido(2S)-GlcNS(6S)-Ido(2S)-GlcNS(6S)-Ido(2S)-GlcNS(6S).

In some implementations, the viral infection is caused by one or more viruses selected from the group consisting of respiratory syncytial virus (RSV), influenza virus, herpes simplex viruses 1 and 2, cytomegalovirus (CMV), vaccinia virus, vesicular stomatitis virus (VSV), Sindbis virus, HIV, human papillomavirus (HPV), influenza A virus, and alphaviruses.

In one aspect, the present technology provides for a kit, which includes a first container, the first container having at least one sulfated polysaccharide, wherein the sulfated polysaccharide includes between 3 to 10 monosaccharide units. In some implementations, the kit includes a second container, the second container having one or more lipids. In some implementations, the kit includes a third container, the third container having a mixture of lipids. In some implementations, the kit further includes a fourth container, the fourth container having at least one carrier molecule or support substrate. In some implementations, the kit includes instructions for making at least one glycolipid, at least one liposome displaying sulfated polysaccharides, or at least one carrier molecule or support substrate displaying sulfated polysaccharides.

In some implementations, the sulfated polysaccharide of the kit include between 1-4 sulfur atoms per disaccharide. In some implementations, the sulfated polysaccharide of the kit includes an octasaccharide, wherein the octasaccharide includes three sulfur atoms per disaccharide. In some implementations, the sulfur atoms are in the form of a sulfate.

In some implementations, the sulfated polysaccharides of the kit include at least one monosaccharide select from the group consisting of Ido(2S), Iduronate, 2-O sulfo-iduronate, and 2,3-split uronic acid. In some implementations, the sulfated polysaccharide of the kit is GlcUA(2S)-GlcNS(6S)-Ido(2S)-GlcNS(6S)-Ido(2S)-GlcNS(6S)-Ido(2S)-GlcNS(6S).

In one aspect, the present technology provides for a kit including at least one glycolipid, wherein the glycolipid includes at least one sulfated polysaccharide linked to a lipid and wherein the sulfated polysaccharide includes between 3 to 10 monosaccharide units.

In some implementations, the kit also includes a liposome, wherein the liposome includes at least one glycolipid and a mixture of lipids, wherein the sulfated polysaccharide is displayed on a surface of the liposome.

In some implementations, the sulfated polysaccharide of the kit includes between 1-4 sulfur atoms per disaccharide. In some implementations, the sulfated polysaccharide of the kit includes an octasaccharide, wherein the octasaccharide includes three sulfur atoms per disaccharide. In some implementations, the sulfur atoms are in the form of a sulfate.

In some implementations, the sulfated polysaccharide of the kit includes at least one monosaccharide select from the group consisting of Ido(2S), Iduronate, 2-O sulfo-iduronate, and 2,3-split uronic acid. In some implementations, the sulfated polysaccharide of the kit is GlcUA(2S)-GlcNS(6S)-Ido(2S)-GlcNS(6S)-Ido(2S)-GlcNS(6S)-Ido(2S)-GlcNS(6S).

In one aspect, the present technology provides for a composition including a sulfated polysaccharide, wherein the polysaccharide comprises between 3 to 10 monosaccharide units. In some implementations, the sulfated polysaccharide includes an octasaccharide, and wherein the octasaccharide includes three sulfur atoms per disaccharide.

### BRIEF DESCRIPTION OF DRAWINGS

FIG. 1 is a diagram showing the molecular structure of a sulfated octasaccharide of formula I.

FIG. 2A is a schematic showing the digestion of heparin with heparin sulfate

FIG. 2B is a chart showing the different sulfated polysaccharides isolated by size exclusion chromatography.

FIG. 2C is a diagram showing the purification of a sulfated octasaccharide.

FIG. 3 is a graph comparing the efficacy of heparin sodium and a sulfated octasaccharide derived from heparin sulfate in blocking infection of MDCK cells by RSV.

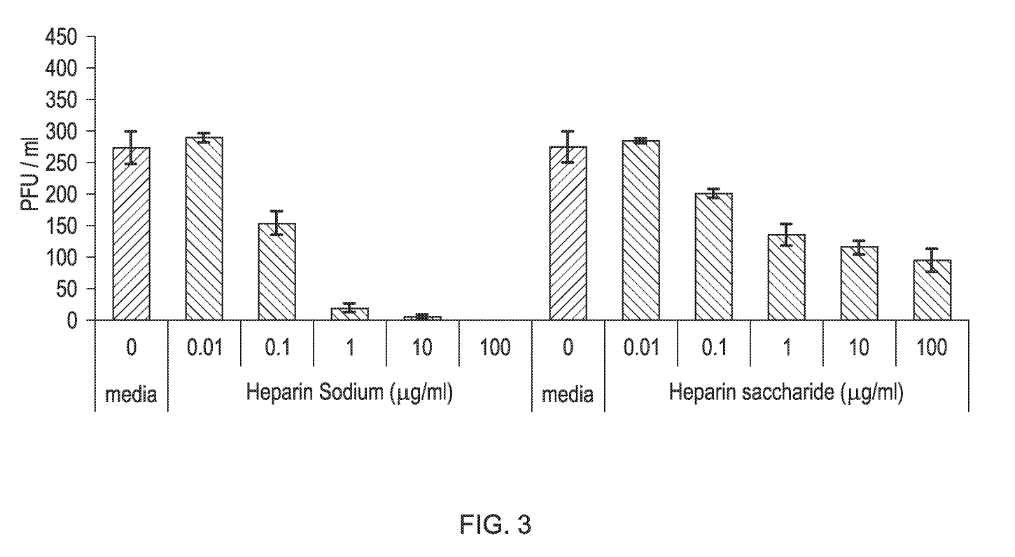

FIG. 4. is a schematic showing the process of amino-oxy synthesis for linking of a phospholipid to a sulfated octasaccharide.

FIG. 5A is graph comparing the infectivity rate of RSV in solution with a sulfated octasaccharide in solution. The sulfated octasaccharide was purified from heparin sulfate.

FIG. 5B is graph comparing the infectivity rate of RSV in solution with a sulfated octasaccharide linked to a phospholipid and incorporated into a liposome. The sulfated octasaccharide was purified from heparin sulfate.

FIG. 6 (A-C) are diagrams showing non-limiting, exemplary molecular structures of sulfated octasaccharides with exemplary R groups. R=—H or —SO₃⁻; R₂=—SO₃⁻ or —Ac.

FIG. 7 provides an exemplary schematic for synthesizing EG3-DOPE, which is a composition including a linker, e.g., EG3 (Boc-EG3-SU) linked to a lipid, e.g., DOPE.

FIG. 8 provides an exemplary schematic for linking a sulfated octasaccharide to EG3-DOPE.

### DETAILED DESCRIPTION

The various concepts introduced above and discussed in greater detail below can be implemented in any of numerous ways, as the described concepts are not limited to any particular manner of implementation. Examples of specific implementations and applications are provided primarily for illustrative purposes. The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a cell” includes a combination of two or more cells, and the like.

As used herein, the “administration” of an agent, drug, or peptide to a subject includes any route of introducing or delivering to a subject a compound to perform its intended function. Administration can be carried out by any suitable route, including orally, intranasally, or parenterally (intravenously, intramuscularly, intraperitoneally, or subcutaneously). Administration includes self-administration and the administration by another.

As used herein, the term “effective amount” refers to a quantity sufficient to achieve a desired therapeutic and/or prophylactic effect, e.g., an amount, which results in the decrease of viral infection in a subject. In the context of therapeutic or prophylactic applications, in some implementations, the amount of a composition administered to the subject will depend on the levels of virus in the subject, and on the characteristics of the individual, such as general health, age, sex, body weight and tolerance to drugs. In some implementations, it will also depend on the degree, severity, and type of disease. The skilled artisan will be able to determine appropriate dosages depending on these and other factors. The compositions can also be administered in combination with one or more additional therapeutic compounds. In the methods described herein, the sulfated polysaccharide and/or sulfated polysaccharide composition can be administered to a subject having one or more signs or symptoms of viral infection such as, e.g., sneezing, muscle aches, fever, congestion (sinus or bronchial), coughing, bronchiolitis, and diminished appetite. For example, a “therapeutically effective amount” of the sulfated polysaccharide and/or sulfated polysaccharide composition includes amounts in which the level of virus is reduced in a subject after administration compared to control subjects who do not receive the compositions. In some implementations, a therapeutically effective amount also reduces or ameliorates the physiological effects, signs or symptoms (e.g., fever, cough, sinus congestion, muscle aches, etc.) of viral infection.

An “isolated” or “purified” sulfated polysaccharide is substantially free of cellular material or other contaminates from the source from which the sulfated polysaccharide is derived, or substantially free from chemical precursors or other chemicals when chemically synthesized. For example, an isolated sulfated polysaccharide would be free of materials that would interfere with diagnostic or therapeutic uses of the agent. Such interfering materials may include enzymes, hormones and other proteinaceous and nonproteinaceous materials.

As used herein, the term “polysaccharide” refers to two or more monosaccharides or multi-saccharides (e.g., sugars) joined to each other by glycosidic bonds. Polysaccharide refers to both short saccharide chains, e.g., saccharide chains less than ten monosaccharide units, and longer saccharide chains, e.g., more than ten monosaccharide units. Polysaccharides include polysaccharide chains modified either by natural processes or by chemical modification techniques that are well known in the art. By way of example, but not by limitation, in some implementations, the polysaccharide includes a disaccharide, trisaccharide, tetrasaccharide, pentasaccharide, hexasaccharide, heptasaccharide, octasaccharide, nonasaccharide, and decasaccharide. The polysaccharide may be branched or straight chain. The polysaccharides of the present disclosure may be formed from monosaccharides (e.g., glucose, fructose, galactose, xylose, ribose, dioses, trioses, tetroses, pentoses, hexoses, heptoses, etc.), disaccharides (e.g., sucrose, lactose, maltose, trehalose, cellobiose, etc.), combinations of mono- and disaccharides, and other forms of saccharides (e.g., multi-saccharides, monosaccharides in the D or L configuration, isomers, stereoisomers, sugar acids such as iduronic acid and the like). By way of example, but not by way of limitation, exemplary saccharides (sugars) useful in the present compositions and methods and their standard abbreviations are presented in the Table 1 below.

TABLE 1

| Exemplary Sugars | Abbreviation |
|---|---|
| Glucose | Glc |
| Galactose | Gal |
| Mannose | Man |
| Rhamnose | Rha |
| Fucose | Fuc |
| Glucosamine | GlcN |
| Galactosamine | GalN |
| Mannosamine | ManN |
| N-acetylglucosamine | GlcNAc |
| N-acetylgalactosamine | GalNAc |
| N-acetylmannosamine | ManNAc |
| Glucuronic acid | GlcA |
| Galacturonic acid | GalA |
| Mannuronic acid | ManA |
| *N-acetylneuraminic acid | NeuNAc |
| *3-deoxy-D-manno-2-octulosonic acid | Kdo |
| Iduronic acid | Ido |
| 2-O sulfo-iduronate | |
| β-D-glucouronic acid | GlcUA |
| 2-O-sulfo-β-D-glucuronic acid | GlcUA(2S) |
| α-L-iduronic acid | IdoUA |
| 2-O-sulfo-α-L-iduronic acid | Ido (2S) |
| β-D-galactose | Gal |
| 6-O-sulfo-β-D-galactose | Gal(6S) |
| β-D-N0acetylgalactsamine | GalNAc |
| β-D-N-acetylgalactosamine-4-O-sulfate | GalNAc(4S) |
| β-D-N-acetylgalactosamine-6-O-sulfate | GalNAc(6S) |
| β-D-N-acetylgalactosamine-4-O, 6-O- | GalNAc(4S,6S) |
| sulfate | |
| α-D-N-acetylglucoasmine | GlcNAc |
| α-D-N-sulfoglucosamine | GlcNS |
| α-D-N-sulfoglucosamine-6-O-sulfate | GlcNS(6S) |
| 2,3-split uronic acid | sU |

As used herein, the term “sulfated polysaccharide” refers to a polysaccharide having at least 1 sulfur atom linked to the polysaccharide. By way of example, but not by limitation, in some implementations, a sulfated polysaccharide has 2, 3, 4, 5, 6, 7, 8, 9, 10, 11, 12, 15, 20 or more sulfur atoms linked to the polysaccharide.

In some implementations, the sulfur atoms are configured on the polysaccharide to allow binding of the polysaccharide to the F and/or G protein of RSV. In some implementations, the sulfur atoms are configured to optimize binding of the polysaccharide to the F and/or G proteins of RSV.

By way of example, but not by limitation, in some implementations, the sulfated polysaccharide includes 1 sulfur atom per monosaccharide, 2 sulfur atoms per monosaccharide, three sulfur atoms per monosaccharide, or 4 sulfur atoms per monosaccharide.

In some implementations, the number of sulfur atoms per monosaccharide (e.g., in a di-, tri-, tetra-, octa- etc. saccharide) can be the same or different. For example, in some implementations, the sulfated polysaccharide comprises 4, 7, 8 or 9 monosaccharide units, with a total of 12, 13, 14, 15, 16 or 17 sulfur atoms on the polysaccharide. For example, the nonosaccharide may be configured to have three sulfur atoms per disaccharide and two sulfur atoms on the remaining monosaccharide.

In some implementations, the sulfated polysaccharide includes 1 sulfur atom per disaccharide, 2 sulfur atoms per disaccharide, 3 sulfur atoms per disaccharide, 4 sulfur atoms per disaccharide or 5 sulfur atoms per disaccharide. By way of example, but not by way of limitation, in some implementations, the sulfated polysaccharide comprises an octasaccharide, having 3 sulfur atoms per disaccharide. As used herein, the term “disaccharide” refers to two linked monosaccharide units. In some implementations, the number of sulfur atoms per disaccharide can be the same or different.

In some implementations, the sulfated polysaccharide includes 1 sulfur atom per trisaccharide, 2 sulfur atoms per trisaccharide, 3 sulfur atoms per trisaccharide, 4 sulfur atoms per trisaccharide, 5 sulfur atoms per trisaccharide, 5 sulfur atoms per trisaccharide, 6 sulfur atoms per trisaccharide, or 7 sulfur atoms per trisaccharide. As used herein the term “trisaccharide” refers to three linked monosaccharide units. In some implementations, the number of sulfur atoms per trisaccharide can be the same or different.

In some implementations, the sulfated polysaccharide includes between 1 to about 16 sulfur atoms per tetrasaccharide. In some implementations, the sulfated polysaccharide includes between 1 to about 20 sulfur atoms per pentasaccharide. In some implementations, the sulfated polysaccharide includes between 1 to 24 sulfur atoms per hexasaccharide. In some implementations, the sulfated polysaccharide includes between 1 to 28 sulfur atoms per heptasaccharide. In some implementations, the sulfated polysaccharide includes between 1 to 32 sulfur atoms per octasaccharide. In some implementations, the sulfated polysaccharide includes between 1 to 36 sulfur atoms per nonasaccharide. In some implementations, the sulfated polysaccharide includes between 1 to 40 sulfur atoms per decasaccharide. By way of example, but not by way of limitation, in some implementations, the sulfated polysaccharide includes an octasaccharide including three sulfur atoms per disaccharide. As noted above, in some implementations, the number of sulfur atoms on each monosaccharide can be the same or different.

In some implementations, the polysaccharide is a trisaccharide having 3 sulfur atoms per disaccharide and between 1-3 sulfur atoms on the remaining monosaccharide. In some implementations, the polysaccharide is a heptasaccharide having 3 sulfur atoms per disaccharide and between 1-3 sulfur atoms on the remaining monosaccharide. In some implementations, the polysaccharide is a nonasaccharide having 3 sulfur atoms per disaccharide and between 1-3 sulfur atoms on the remaining monosaccharide.

As used herein, “glycolipid” refers to a polysaccharide linked to a lipid. As used herein, “lipid” has its customary meaning in the art and refers, for example, to any synthetic, semi-synthetic, or naturally occurring lipids, including phospholipids, tocopherols, sterols, fatty acids, glycoproteins (e.g., albumin), negatively charged lipids, and cationic lipids. In some implementations, the polysaccharide is a sulfated polysaccharide. By way of example, but not by limitation, in some implementations, a glycolipid includes a sulfated octasaccharide linked to a phospholipid.

As used herein, the term “linked composition” refers to at least two (e.g., 2, 3, 4, a plurality of) sulfated polysaccharides, and/or glycolipids, which are linked to the surface of a carrier molecule or support substrate. Examples of carrier molecules or support substrates include, but are not limited to, peptides, polypeptide, protein, carbohydrate, nanoparticles (e.g., metal, polymeric), polymers (e.g., polymer beads), glass beads, magnetic particles, nucleic acid, small molecule, cells, or virus. In some implementations, the solid support is non-toxic to a mammalian subject, is biodegradable and/or can be broken down and/or absorbed by the subject's body. In some implementations, the carrier molecule or support substrate includes one or more of a polymer, dendrimer, particle, bead, macromolecule, and solid lipid particle. By way of example, but not by way of limitation, in some implementations, a linked composition includes two sulfated octasaccharides linked to a bead, wherein the octasaccharides includes at least three sulfur atoms per disaccharide.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients.

As used herein, the terms “treating” or “treatment” or “alleviation” refers to therapeutic treatment, wherein the object is to prevent, alleviate, ameliorate or slow down (lessen) the targeted pathologic condition or disorder. For example, a subject is successfully “treated” for viral infection if, after receiving a therapeutic amount of the sulfated polysaccharide composition according to the methods described herein, the subject shows observable and/or measurable reduction in or absence of one or more signs and symptoms of viral infection, such as, e.g., fever, cough, sinus congestion, muscle aches, and diminish appetite. It is also to be appreciated that the various modes of treatment or prevention of medical conditions as described are intended to mean “substantial,” which includes total but also less than total treatment or prevention, and wherein some biologically or medically relevant result is achieved.

As used herein, “prevention” or “preventing” of an infection refers to a compound that, in a statistical sample, reduces the occurrence of viral infection in the treated sample relative to an untreated control sample, or delays the onset or reduces the severity of one or more symptoms of the viral infection relative to the untreated control sample. As used herein, preventing viral infection includes preventing the initiation of viral entry into a cell, preventing a virus from binding to a cell, delaying the infection of a cell by the virus, preventing the progression or advancement of viral infection, and reversing the progression of viral infection from an advanced to a less advanced stage.

### I. Sulfated Polysaccharides and Compositions Thereof

A. General

The present technology relates to the treatment or prevention of viral infection, e.g., respiratory syncytial virus (“RSV”) infection, by administration of compositions including a sulfated polysaccharide of the present disclosure, to a subject in need thereof. The compositions typically bind the virus (e.g., RSV) with high affinity. The compositions can be administered to humans or animals infected with virus to treat infection, or given to those exposed to virus or likely to be exposed to virus, or those at risk of infection, thereby to prevent infection.

Previous studies have demonstrated that cell surface heparan sulfate (“HS”) plays an important role in infection by RSV. HS is a highly sulfated, linear polysaccharide that is present on the surface of mammalian cells and the extracellular matrix. It has a number of biological activities, and the specific sulfation and saccharide sequences play an important role in determining the function of HS. Heparin is a specific form of heparan sulfate that has significant anticoagulant activity, and is used for therapeutic purposes.

The RSV virion interacts directly with cell surface HS, and binding to HS is mediated by both the F and G envelope glycoproteins of RSV. Attachment and infectivity of RSV strains are diminished in the presence of soluble heparan sulfate, and removal of cell surface heparan sulfate substantially limits infectivity.

A detailed structural analysis of the RSV F and G proteins has identified a potential heparan sulfate binding site. The G protein binding motif is subgroup dependent (subgroup A, A184 to T198, and subgroup B, K183 to K197), but constitutes a short, linear peptide that involves a secondary interaction that likely “bridges” with the primary interaction with the F protein. The F protein contains a more extended multi-domain set of features, including F1, represented by K201→I217 and the consensus sequences L257→S287, K327→C343, and S404→T434 and F2, represented by Y33→R49 and the consensus sequence T54→K77. The sulfated polysaccharide compositions of the present disclosure block RSV infection with greater efficacy than heparin sulfate.

The sulfated polysaccharide compositions of the present disclosure act by binding to the virus and preventing viral adherence to, and interaction with, cells within the body. In some implementations, binding efficacy is improved when the sulfated polysaccharides are attached to a suitable solid support or carrier molecule (e.g., polymer beads) or provided in the form of liposomes, thereby facilitating the interaction of the virus with multiple sulfated polysaccharides simultaneously.

B. Compositions

The compositions of the present disclosure include at least one sulfated polysaccharide. In some implementations, the compositions include at least one glycolipid, wherein the glycolipid includes a sulfated polysaccharide. Additionally or alternatively, in some implementations, the sulfated polysaccharide and/or the glycolipid are in the form of a liposome. Additionally or alternatively, in some implementations, the sulfated polysaccharide and/or the glycolipid are linked to a solid support or carrier molecule (e.g., polymer beads).

1. Sulfated Polysaccharide

In some implementations, the sulfated polysaccharide binds to the virus's F envelope glycoprotein, G envelope glycoprotein, or both. By binding to the F and/or G envelope glycoproteins, the sulfated polysaccharides of the present disclosure prevent viral binding to cell surface heparan sulfate, thereby by preventing viral entry into cells. By way of example, but not by way of limitation, the sulfated polysaccharide is configured such that the sulfates interact with the positive charged amino acids on the F protein. In some implementations, each monomer of the polysaccharide is fully sulfated. In some implementations, each monomer (e.g., monosaccharide) of the polysaccharide includes 1, 2 or 3 sulfates. In some implementations, each disaccharide of the polysaccharide includes 1, 2 or 3 sulfates. In some implementations, the sulfated polysaccharide includes monosaccharides, which provide conformational flexibility to the polysaccharide as a whole, facilitating contact of the sulfates with the F and G protein active sites. By way of example, but not by way of limitation, monomers such as Ido (2S), iduronate, 2-O sulfo-iduronate, or 2,3-split uronic acid (sU) are employed to provide such flexibility. The sulfated polysaccharides of the present disclosure are not intended to be limited by the monosaccharides or sugars used to form the polysaccharide chain, or the methods used to form the polysaccharide chain. By way of example, but not by way of limitation, in some implementations, the sulfated polysaccharide is formed from a combination of GlcUA (2S), GlcNS (6S), and Ido (2S) monomers.

In some implementations, the sulfated polysaccharides of the compositions disclosed herein are the same (e.g., include the same saccharide sequence and sulfanation profile). In some implementations, the compositions include a mixture of different sulfated polysaccharides. In some implementations, the sulfated polysaccharide includes 3, 4, 5, 6, 7, 8, 9, or 10 monosaccharide units. In some implementations, the composition includes a sulfated polysaccharide including 6-10 monosaccharide units, or about 8 monosaccharide units.

In some implementations, the sulfated polysaccharide includes between 1 to 4 or 2-3 sulfur atoms per monosaccharide. In some implementations, the sulfated polysaccharide includes between 1 to 8 sulfur atoms, between 3-6, or 3 sulfur atoms per disaccharide. In some implementations, the sulfated polysaccharide includes between 1 to 10 sulfur atoms, between 3 to 8 sulfur atoms, or 7 sulfur atoms per trisaccharide. In some implementations, the sulfated polysaccharide includes between 1 to 12 sulfur atoms, between 3 to 10 sulfur atoms, or between 5 to 7 per tetrasaccharide. In some implementations, the sulfated polysaccharide includes between 1 to 14 sulfur atoms, between 3 to 11 sulfur atoms, between 5 to 9 sulfur atoms per pentasaccharide. In some implementations, the sulfated polysaccharide includes between 1 to 16 sulfur atoms, between 3 to 14 sulfur atoms, between 6 to 11 sulfur atoms, or between 7 to 9 sulfur atoms per hexasaccharide. In some implementations, the sulfated polysaccharide includes between 1 to 18 sulfur atoms, between 3 to 15 sulfur atoms, between 6 to 12 sulfur atoms, or between 8 to 10 sulfur atoms per heptasaccharide. In some implementations, the sulfated polysaccharide includes between 1 to 20 sulfur atoms, between 3 to 18 sulfur atoms, between 6 to 15 sulfur atoms, or between 9 to 12 sulfur atoms per octasaccharide. In some implementations, the sulfated polysaccharide includes between 1 to 22 sulfur atoms, between 3 to 19 sulfur atoms, between 6 to 16 sulfur atoms, between 8 to 14 sulfur atoms, or between 10 to 12 sulfur atoms per nonasaccharide. In some implementations, the sulfated polysaccharide includes between 1 to 24 sulfur atoms, 3 to 21 sulfur atoms, between 6 to 18 sulfur atoms, between 9 to 15 sulfur atoms, or between 11 to 13 sulfur atoms per decasaccharide.

By way of example, but not by way of limitation, in some implementations, the sulfated polysaccharide includes octasaccharide including three sulfur atoms per disaccharide. By way of example, but not by way of limitation, in some implementations, the sulfated polysaccharide includes a tetrasaccharide, a heptasaccharide, or a decasaccharide having three sulfur atoms per disaccharide, and between 1-3 sulfur atoms on the remaining monosaccharide.

In some implementations, the sulfated polysaccharide includes sulfur atoms in a ratio of one sulfur atom, two sulfur atom, three sulfur atoms or four sulfur atom per two monosaccharide units. In some implementations, the sulfated polysaccharide includes sulfur atoms in a ratio of three sulfur atoms per two monosaccharide units. In some implementations, the sulfated polysaccharide includes sulfur atoms in a ratio of one sulfur atom, two sulfur atom, three sulfur atoms, four sulfur atom, five sulfur atom, or six sulfur atoms per three monosaccharide units. In some implementations, the sulfated polysaccharide includes sulfur atoms in a ratio of one sulfur atom, two sulfur atom, three sulfur atoms, four sulfur atom, five sulfur atom, six sulfur atoms, seven sulfur atom, or eight sulfur atoms per four monosaccharide units. In some implementations, the sulfated polysaccharide includes sulfur atoms in a ratio of one sulfur atom, two sulfur atom, three sulfur atoms, four sulfur atom, five sulfur atom, six sulfur atoms, seven sulfur atom, eight sulfur, nine sulfur atoms, or ten sulfur atoms per five monosaccharide units. In some implementations, the above ratios of sulfur atoms to monosaccharide units can be combined for polysaccharide chains greater than five monosaccharides, e.g., heptasaccharide may have at most a ratio of twenty-eight sulfur atoms per seven monosaccharides.

In any of the disclosed implementations, the sulfur atom may be present in the form of a sulfate.

In some implementations, the sulfated polysaccharide includes between 1 to 4 or 2-3 sulfates per monosaccharide. In some implementations, the sulfated polysaccharide includes between 1 to 8 sulfates, between 3-6, or 3 sulfates per disaccharide. In some implementations, the sulfated polysaccharide includes between 1 to 10 sulfates, between 3 to 8 sulfates, or 7 sulfates per trisaccharide. In some implementations, the sulfated polysaccharide includes between 1 to 12 sulfates, between 3 to 10 sulfates, or between 5 to 7 per tetrasaccharide. In some implementations, the sulfated polysaccharide includes between 1 to 14 sulfates, between 3 to 11 sulfates, between 5 to 9 sulfates per pentasaccharide. In some implementations, the sulfated polysaccharide includes between 1 to 16 sulfates, between 3 to 14 sulfates, between 6 to 11 sulfates, or between 7 to 9 sulfates per hexasaccharide. In some implementations, the sulfated polysaccharide includes between 1 to 18 sulfates, between 3 to 15 sulfates, between 6 to 12 sulfates, or between 8 to 10 sulfates per heptasaccharide. In some implementations, the sulfated polysaccharide includes between 1 to 20 sulfates, between 3 to 18 sulfates, between 6 to 15 sulfates, or between 9 to 12 sulfates per octasaccharide. In some implementations, the sulfated polysaccharide includes between 1 to 22 sulfates, between 3 to 19 sulfates, between 6 to 16 sulfates, between 8 to 14 sulfates, or between 10 to 12 sulfates per nonasaccharide. In some implementations, the sulfated polysaccharide includes between 1 to 24 sulfates, 3 to 21 sulfates, between 6 to 18 sulfates, between 9 to 15 sulfates, or between 11 to 13 sulfates per decasaccharide.

By way of example, but not by limitation, in some implementations the sulfated polysaccharide includes a sulfated octasaccharide with three sulfates per disaccharide. By way of example, but not by way of limitation, in some implementations, the sulfated polysaccharide includes a tetrasaccharide, or a heptasaccharide, or a decasaccharide having three sulfates per disaccharide.

In some implementations, the sulfated polysaccharide includes sulfate in a ratio of one sulfate, two sulfates, three sulfates, or four sulfates per two monosaccharide units. In some implementations, the sulfated polysaccharide includes sulfate in a ratio of one sulfate, two sulfates, three sulfates, four sulfates, five sulfates, or six sulfates per three monosaccharide units. In some implementations, the sulfated polysaccharide includes sulfate in a ratio of one sulfate, two sulfates, three sulfates, four sulfates, five sulfates, six sulfates, seven sulfates, or eight sulfates per four monosaccharide units. In some implementations, the sulfated polysaccharide includes sulfate in a ratio of one sulfate, two sulfates, three sulfates, four sulfates, five sulfates, six sulfates, seven sulfates, eight sulfates, nine sulfates, or ten sulfates per five monosaccharide units. In some implementations, the above ratios sulfate to monosaccharide units can be combined for polysaccharide chains greater than five monosaccharides, e.g., heptasaccharide may have at most a ratio of fourteen sulfates per seven monosaccharides.

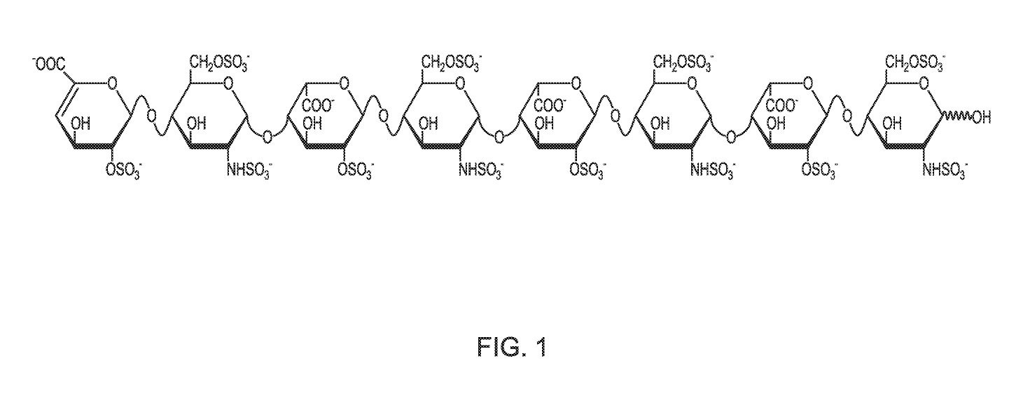

By way of example, but not by way of limitation, in some implementations, the sulfated polysaccharide includes one or more of GlcUA(2S)-GlcNS(6S)-Ido(2S)-GlcNS(6S)-Ido(2S)-GlcNS(6S)-Ido(2S)-GlcNS(6S) (FIG. 1), GlcUA(2S)-GlcNS(6S)-sU-GlcNS(6S)-sU-GlcNS(6S)-sU-GlcNS(6S), and GlcUA(2S)-GlcNS-Ido(2S)-GlcNS-Ido(2S)-GlcNS-Ido(2S)-GlcNS.

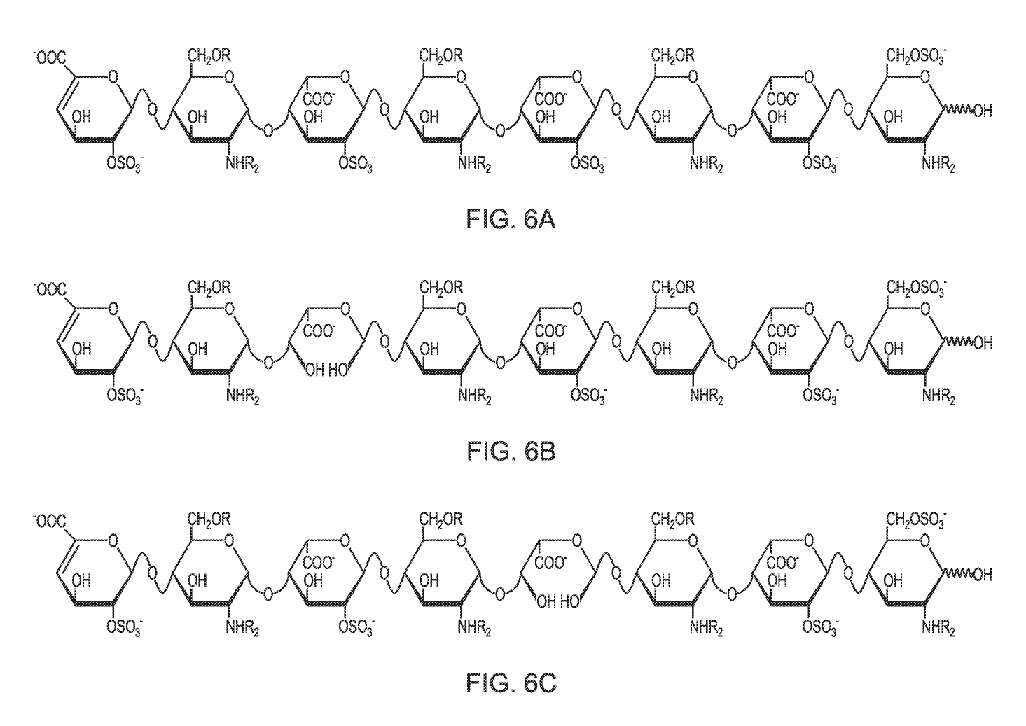

In some implementations, the sulfated polysaccharide includes one or more substitutions on one more monosaccharides at an R position. In some implementations, the sulfated polysaccharide includes at most five substitutions. Substitutions include, but are not limited to, esters, acetyl, —H, or —SO₃⁻. Non-limiting examples of locations of R positions are depicted in FIG. 6 A-C.

2. Compositions Providing Multivalent Display of Sulfated Polysaccharide

In some implementations, multivalent display is achieved by having at least two sulfated polysaccharide (e.g., a plurality) displayed on a carrier molecule or support substrate, or provided on the surface of a liposome.

In some implementations, the sulfated polysaccharide is linked to a lipid to form a glycolipid. In some implementations, at least one glycolipid (e.g., 2, 3, 4, or plurality) is combined with a mixture of additional lipids to form liposomes. Additionally, or alternatively, in some implementations, at least two sulfated polysaccharide (e.g., 2, 3, 4, or plurality), and/or at least one glycolipid (e.g., 2, 3, 4, or plurality) is linked to the surface of a carrier molecule or support substrate (hereinafter “linked composition”). In some implementations, the liposome and/or the linked composition improves the binding efficiency of the sulfated polysaccharide to a virion or virions by facilitating multivalent interactions with the virion or virions.

In some implementations, the lipids linked to the sulfated polysaccharide include, but are not limited to, synthetic, semi-synthetic, or naturally occurring lipids, including phospholipids, tocopherols, sterols, fatty acids, glycoproteins (e.g., albumin), negatively charged lipids, and cationic lipids.

Phospholipids include, but are not limited to, sphingomyelin, phosphatidic acid (e.g., DMPA, DPPA, and DSPA), phosphatidylcholine (e.g., DDPC, DLPC, DMPC, DPPC, DSPC, DOPC, POPC, and DEPC), phosphatidylglycerol (e.g., DMPG, DPPG, DSPG, and POPG), phosphatidylinositol, phosphatidylethanolamine (e.g., DMPE, DPPE, DSPE, and DOPE), phosphatidylserine (e.g., DOPS), the egg counterparts of the above listed phospholipids (e.g., egg phosphatidylcholine (EPC), egg phosphatidylglycerol (EPG), egg phosphatidylinositol (EPI), egg phosphatidylserine (EPS), phosphatidylethanolamine (EPE)), the soy counterparts of the above listed phospholipids (e.g., soy phosphatidylcholine (SPC), SPG, SPS, SPI, SPE, and SPA), the hydrogenated egg and soy counterparts of the above listed phospholipids (e.g., HEPC, HSPC), PEG phospholipid (e.g., mPEG-phospholipid, poly-glycerin-phospholipid, and functionalized-phospholipid), phosphoinositides (e.g., phosphoatidylinositol, phosphoatidylinositol phosphate, phosphoatidylinositol biphosphate, and phosphoatidylinositol triphosphate), mixed phospholipids like palmitoylstearoylphosphatidyl-choline (PSPC), and palmitoylstearolphosphatidylglycerol (PSPG), and single acylatedphospholipidslikemono-oleoyl-phosphatidyletha nolamine (MOPE), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids, or a combination thereof. The chains on these fatty acids can be saturated or unsaturated, and the phospholipid may be made up of fatty acids of different chain lengths and different degrees of unsaturation.

Sterols include, but are not limited to, cholesterol, esters of cholesterol (including cholesterol hemi-succinate), salts of cholesterol (including cholesterol hydrogen sulfate and cholesterol sulfate), ergosterol, esters of ergosterol (including ergosterol hemi-succinate), salts of ergosterol (including ergosterol hydrogen sulfate and ergosterol sulfate, lanosterol, esters of lanosterolincludinglanosterolhemi-succinate, salts of lanosterol including lanosterol hydrogen sulfate and lanosterol sulfate.

Tocopherols include, but are not limited to, tocopherols, esters of tocopherols (including tocopherol hemi-succinates), salts of tocopherols (including tocopherol hydrogen sulfates and tocopherol sulfates).

Cationic lipids include, but are not limited to, ammonium salts of fatty acids, phospholipids and glycerides. Examples of cationic lipids include, but are not limited to myristylamine, palmitylamine, laurylamine, stearylamine, dilauroyl ethylphosphocholine (DLEP), dimyristoyl ethylphosphocholine (DMEP), dipalmitoylethylphosphocholine(DPEP), and distearoylethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio) propane (DOTAP).

Negatively-charged lipids include, but are not limited to, phosphatidylglycerol, phosphatidic acid, phosphatidylinositols, and phosphatidylserines.

In some implementations, additional lipids not linked to the sulfated polysaccharide are included in the compositions to form liposomes. In some implementations, the additional lipids include, but are not limited to, any of the above listed lipids.

As noted previously, in some implementations, multivalent display is achieved by having at least two sulfated polysaccharides or two glycolipids displayed on a carrier molecule or support substrate. By way of example, but not by way of limitation, carrier molecules or support substrates include, but are not limited to, peptides, polypeptide, protein, carbohydrate, nanoparticles (e.g., metal, polymeric), linear polymers, polymer beads, dendrimers, glass beads, magnetic particles, nucleic acid, small molecule, cells, or virus. In some implementations, the solid support is non-toxic to a mammalian subject, is biodegradable and/or can be broken down and/or absorbed by the subject's body. In some implementations, the carrier molecule or support substrate includes one or more of a polymer, dendrimer, particle, bead, macromolecule, and solid lipid particle.

In some implementations, the liposomes or the linked compositions are more effective in inhibiting viral infection than the sulfated polysaccharide alone. In some implementations, the liposome or linked compositions are about 100-fold, 200-fold, 400-fold, 800-fold, 1000-fold, 1500-fold, 2000-fold more effective in inhibiting viral infection than the sulfated polysaccharide alone.

### II. Methods for Making the Sulfated Polysaccharide Composition

A. Methods for Making a Sulfated Polysaccharide

In some implementations, formation of a sulfated polysaccharide includes isolating at least one sulfated polysaccharide from heparin or heparan sulfate. By way of example, but not by limitation, in some implementations, isolation of sulfated polysaccharide includes: 1) digesting heparin or heparan sulfate with heparinase; 2) size-fractionation of the digestion product by size exclusion chromatography; 3) isolating the pool of the desired sulfated polysaccharide chain length (e.g., isolating the sulfated octasaccharide pool); and 4) subjecting the isolated sulfated polysaccharide pool to strong-anion exchange to isolate and purify the desired sulfated polysaccharide (e.g., isolating and purify an octasaccharide that binds to virion).

In some implementations, the sulfated polysaccharide is produced synthetically. Any methods known in the art to produce the sulfated polysaccharide may be used for example, see DeAngelis et al., Glycobiology. 23(7); 764-77 (July 2013).

B. Methods for Making Liposomes

General

By way of example, but not by limitation, in some implementations, formation of a liposome includes: 1) attaching at least two sulfated polysaccharides, e.g., as described above, to a lipid to form a glycolipid; 2) combining at least one glycolipid with other lipids to form a lipid mixture; and 3) forming liposomes by pressing the lipid mixture through a membrane.

Methods for Making Glycolipids

Non-limiting examples of lipids used in the formation of the glycolipid are listed above. In some implementations, the sulfated polysaccharide and lipid are mixed and incubated between about 20° C. to about 25° C. In some implementations, the sulfated polysaccharide and lipid are mixed in a buffer containing about 1% to about 5% acetic acid and THF.

By way of example, but not by limitation, in some implementations, methods useful for linking lipids to the sulfated polysaccharide include: 1) Reductive amination using polar aprotic solvents, e.g., DMSO, DMF or THF, using a high amine to sugar ratio, e.g., about 10-100:1 at a pH of about 4-5; 2) Click chemistry between an azide and an alkyne in biological buffers at a ratio of 1:1; 3) Aminooxy conjugation in biological buffers at a ratio of 1:1 at a pH of about 4-5; 4) Reacting with hydrazide or semicarbazide at a ratio of about 1-5:1 at a pH of about 4-5.

In some implementations, a linker is attached to the lipid, and the sulfated polysaccharide is attached to the lipid via the linker. By way of example, but not by way of limitation, in some implementation, linkers include, but are not limited to, N-Boc-N′-succinyl-4,7,10-trioxa-1,13-tridecanediamine (i.e., Boc-EG3-Su; Chemodex, San Diego, Calif.) and N-(FMoc-13-amino-4,7,10-trioxa-tridecyl)succinamic acid (Polypeptide Laboratories, San Diego, Calif.).

In some implementations, the glycolipids are purified using reverse phase HPLC.

Methods for Making Liposomes

In some implementations, one or more glycolipids are combined with a plurality of at least one other type of lipid to form a lipid mixture that will be used to make liposomes. In some implementations, the additional lipids are mixed in chloroform before mixing with the glycolipid. In some implementations, the glycolipid is in water before mixing with the additional lipids. In some implementations, the water mixture with the glycolipid and the chloroform mixture of additional lipids are combined at room temperature. In some implementations, the lipid mixture includes about 1% to about 30% glycolipids and about 69% to about 99% of additional lipid. In some implementations, the additional lipids in the lipid mixture are the same type of lipid or a mixture of different types of lipids. In some implementations, the mix of additional lipids includes about 1% to about 30%, about 3% to about 27%, about 6% to about 24%, about 9% to about 21%, about 12% to about 18%, or about 14% to about 16% cholesterol. In some implementations, the mix of additional lipids includes about 69% to about 98%, about 72% to about 95%, about 75% to about 92%, about 78% to about 89%, or about 81% to about 86% DOPC.

In some implementations, the lipid mixture includes, in addition to glycolipid, cholesterol, other fatty acid structures (e.g., DSPC instead of DOPC), lipids with other types of headgroups (e.g., DOPG), or adding modified lipids (e.g., PEGylated lipids). Any combinations and formulations of lipid mixtures known in the art for making liposome can be implemented.

The lipids in the mix of lipid include, but are not limited to, synthetic, semi-synthetic, or naturally occurring lipids, including phospholipids, tocopherols, sterols, fatty acids, glycoproteins (e.g., albumin), negatively charged lipids, and cationic lipids.

Phospholipids include, but are not limited to, sphingomyelin, phosphatidic acid (e.g., DMPA, DPPA, and DSPA), phosphatidylcholine (e.g., DDPC, DLPC, DMPC, DPPC, DSPC, DOPC, POPC, and DEPC), phosphatidylglycerol (e.g., DMPG, DPPG, DSPG, and POPG), phosphatidylinositol, phosphatidylethanolamine (e.g., DMPE, DPPE, DSPE, and DOPE), phosphatidylserine (e.g., DOPS), the egg counterparts of the above listed phospholipids (e.g., egg phosphatidylcholine (EPC), egg phosphatidylglycerol (EPG), egg phosphatidylinositol (EPI), egg phosphatidylserine (EPS), phosphatidylethanolamine (EPE)), the soy counterparts of the above listed phospholipids (e.g., soy phosphatidylcholine (SPC), SPG, SPS, SPI, SPE, and SPA), the hydrogenated egg and soy counterparts of the above listed phospholipids (e.g., HEPC, HSPC), PEG phospholipid (e.g., mPEG-phospholipid, poly-glycerin-phospholipid, and functionalized-phospholipid), phosphoinositides (e.g., phosphoatidylinositol, phosphoatidylinositol phosphate, phosphoatidylinositol biphosphate, and phosphoatidylinositol triphosphate), mixed phospholipids like palmitoylstearoylphosphatidyl-choline (PSPC), and palmitoylstearolphosphatidylglycerol (PSPG), and single acylatedphospholipidslikemono-oleoyl-phosphatidyletha nolamine (MOPE), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids, or a combination thereof. The chains on these fatty acids can be saturated or unsaturated, and the phospholipid may be made up of fatty acids of different chain lengths and different degrees of unsaturation.

Sterols include, but are not limited to, cholesterol, esters of cholesterol (including cholesterol hemi-succinate), salts of cholesterol (including cholesterol hydrogen sulfate and cholesterol sulfate), ergosterol, esters of ergosterol (including ergosterol hemi-succinate), salts of ergosterol (including ergosterol hydrogen sulfate and ergosterol sulfate, lanosterol, esters of lanosterolincludinglanosterolhemi-succinate, salts of lanosterol including lanosterol hydrogen sulfate and lanosterol sulfate.

Tocopherols include, but are not limited to, tocopherols, esters of tocopherols (including tocopherol hemi-succinates), salts of tocopherols (including tocopherol hydrogen sulfates and tocopherol sulfates).

Cationic lipids include, but are not limited to, ammonium salts of fatty acids, phospholipids and glycerides. Examples of cationic lipids include, but are not limited to myristylamine, palmitylamine, laurylamine, stearylamine, dilauroyl ethylphosphocholine (DLEP), dimyristoyl ethylphosphocholine (DMEP), dipalmitoylethylphosphocholine(DPEP), and distearoylethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio) propane (DOTAP).

Negatively-charged lipids include, but are not limited to, phosphatidylglycerol, phosphatidic acid, phosphatidylinositols, and phosphatidyl serines.

In some implementations, the glycolipids/additional lipid mixture is extruded through a membrane to form liposomes. In some implementations, the pores in the membrane are about 25 nm, about 50 nm, about 100 nm, about 200 nm, about 300 nm, about 400 nm, about 500 nm, about 600 nm, about 700 nm, about 800 nm, about 900 nm, about 1000 nm, or any ranges between any two of these values. In some implementations, the membrane is an aluminum oxide membrane or a polycarbonate membrane. In some implementations, extrusions through the membrane are between about 5 to 30 passes, or about 10 to 25 passes, or about 15 to 20 passes.

C. Method for Linking a Sulfated Polysaccharide or Glycolipid to a Solid Support

In some implementations, sulfated polysaccharides are bound to a support substrate or carrier molecule by methods that include, but are not limited to, ionic interaction, hydrophobic interaction, or crystallization, or chemical methods such as radical polymerization, aldehyde chemistry, or condensation.

### III. Methods of Using Sulfated Polysaccharide Composition

General.

The sulfated polysaccharides, liposomes, and linked compositions described herein are useful to prevent or treat viral infection. Specifically, the disclosure provides for both prophylactic and therapeutic methods of treating a subject having or at risk of viral infection. Accordingly, the present methods provide for the prevention and/or treatment of viral infection in a subject by administering an effective amount of the sulfated polysaccharides, liposomes, linked compositions, or a combination thereof to a subject in need thereof. By way of example, but not by way of limitation, in some implementations, the sulfated polysaccharide, liposomes, linked compositions, or a combination thereof are able to treat or prevent infection by, but not limited to, respiratory syncytial virus (RSV), herpes simplex viruses 1 and 2, cytomegalovirus (CMV), vaccinia virus, vesicular stomatitis virus (VSV), Sindbis virus, and HIV, human papillomavirus (HPV), influenza A virus, alphaviruses (e.g., Chickungunya, Western equine encephalitis, Eastern equine encephalitis, Venezuelan equine encephalitis), flaviviruses (e.g., dengue and west nile).

Therapeutic Methods.

One aspect of the present technology includes methods of treating viral infection in a subject for therapeutic purposes. In therapeutic applications, compositions or medicaments are administered to a subject suspected of, or already suffering from, a viral infection in an amount sufficient to cure, or at least partially arrest, the symptoms of the disease, including its complications and intermediate pathological phenotypes in development of the disease. As such, the present technology provides methods of treating an individual with a viral infection.

Subjects suffering from viral infection can be identified by any or a combination of diagnostic or prognostic assays known in the art. For example, typical symptoms of viral infection include, but are not limited to, e.g., sneezing, muscle aches, fever, sinus congestion, coughing, bronchiolitis, and diminished appetite.

Prophylactic Methods.

In one aspect, the invention provides a method for preventing, in a subject, viral infection by administering an effective amount of the sulfated polysaccharide, liposomes, linked compositions, or a combination thereof that prevents the initiation or progression of viral infection. Subjects at risk for viral infection can be identified by, e.g., any or a combination of diagnostic or prognostic assays as known in the art. In prophylactic applications, pharmaceutical compositions or medicaments of the sulfated polysaccharide, liposomes, linked compositions, or a combination thereof are administered to a subject susceptible to, or otherwise at risk for viral infection in an amount sufficient to eliminate or reduce the risk, lessen the severity, or delay the progression of the infection. Administration of a prophylactic sulfated polysaccharide, liposomes, linked compositions, or a combination thereof can occur prior to the manifestation of symptoms characteristic of viral infection, such that a viral infection is prevented or, alternatively, delayed in its progression.

Determination of the Biological Effect of the Aromatic-Cationic Peptide-Based Therapeutic.

In various implementations, suitable in vitro or in vivo assays are performed to determine the effect of a specific sulfated polysaccharide, liposome, linked composition, or a combination thereof based therapeutic and whether its administration is indicated for treatment. In various implementations, in vitro assays are performed with representative animal models to determine if a given sulfated polysaccharide, liposome, linked composition, or a combination thereof based therapeutic exerts the desired effect in preventing or treating viral infection. Compounds for use in therapy are tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

### IV. Kits

In some implementations, the compositions of the present technology are provided in a kit.

In some implementations, a kit includes at least one sulfated polysaccharide, at least one liposome, at least one linked composition, or a combination thereof. In some implementations, the sulfated polysaccharide, liposome, or linked compositions are in individual containers. In some implementations, the sulfated polysaccharide, liposome, linked composition are combined together in a single container. In some implementations, the kit also includes tools (e.g., a syringe or nebulizer) for delivery of the sulfated polysaccharide, liposome, linked composition, or combination thereof. In some implementations, the sulfated polysaccharide is isolated or derived from heparin or heparan sulfate. In some implementations, the sulfated polysaccharide, liposome, or linked composition includes formula I (FIG. 1).

In some implementations, a kit includes a first container having at least one sulfated polysaccharide, a second container having at least one first lipid, a third container having at least one second lipid, at least one membrane, and a tool for extrusion of a mixture through the membrane. In some implementations, the sulfated polysaccharide is isolated or derived from heparin or heparan sulfate. In some implementations, the sulfated polysaccharide is formula I (FIG. 1). In some implementations, the membrane has pores that are about 50 nm, about 100 nm, about 150 nm, about 200 nm, about 250 nm, about 300 nm. In some implementations, the kit also includes instructions for making a liposome. In some implementations, the first and second lipid include, but are not limited to, synthetic, semi-synthetic, or naturally occurring lipids, including phospholipids, tocopherols, sterols, fatty acids, glycoproteins (e.g., albumin), negatively charged lipids, and cationic lipids.

Phospholipids include, but are not limited to, sphingomyelin, phosphatidic acid (e.g., DMPA, DPPA, and DSPA), phosphatidylcholine (e.g., DDPC, DLPC, DMPC, DPPC, DSPC, DOPC, POPC, and DEPC), phosphatidylglycerol (e.g., DMPG, DPPG, DSPG, and POPG), phosphatidylinositol, phosphatidylethanolamine (e.g., DMPE, DPPE, DSPE, and DOPE), phosphatidylserine (e.g., DOPS), the egg counterparts of the above listed phospholipids (e.g., egg phosphatidylcholine (EPC), egg phosphatidylglycerol (EPG), egg phosphatidylinositol (EPI), egg phosphatidylserine (EPS), phosphatidylethanolamine (EPE)), the soy counterparts of the above listed phospholipids (e.g., soy phosphatidylcholine (SPC), SPG, SPS, SPI, SPE, and SPA), the hydrogenated egg and soy counterparts of the above listed phospholipids (e.g., HEPC, HSPC), PEG phospholipid (e.g., mPEG-phospholipid, poly-glycerin-phospholipid, and functionalized-phospholipid), phosphoinositides (e.g., phosphoatidylinositol, phosphoatidylinositol phosphate, phosphoatidylinositol biphosphate, and phosphoatidylinositol triphosphate), mixed phospholipids like palmitoylstearoylphosphatidyl-choline (PSPC), and palmitoylstearolphosphatidylglycerol (PSPG), and single acylatedphospholipidslikemono-oleoyl-phosphatidyletha nolamine (MOPE), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids, or a combination thereof. The chains on these fatty acids can be saturated or unsaturated, and the phospholipid may be made up of fatty acids of different chain lengths and different degrees of unsaturation.

Sterols include, but are not limited to, cholesterol, esters of cholesterol (including cholesterol hemi-succinate), salts of cholesterol (including cholesterol hydrogen sulfate and cholesterol sulfate), ergosterol, esters of ergosterol (including ergosterol hemi-succinate), salts of ergosterol (including ergosterol hydrogen sulfate and ergosterol sulfate, lanosterol, esters of lanosterolincludinglanosterolhemi-succinate, salts of lanosterol including lanosterol hydrogen sulfate and lanosterol sulfate.

Tocopherols include, but are not limited to, tocopherols, esters of tocopherols (including tocopherol hemi-succinates), salts of tocopherols (including tocopherol hydrogen sulfates and tocopherol sulfates).

Cationic lipids include, but are not limited to, ammonium salts of fatty acids, phospholipids and glycerides. Examples of cationic lipids include, but are not limited to myristylamine, palmitylamine, laurylamine, stearylamine, dilauroyl ethylphosphocholine (DLEP), dimyristoyl ethylphosphocholine (DMEP), dipalmitoylethylphosphocholine(DPEP), and distearoylethylphosphocholine (DSEP), N-(2,3-di-(9-(Z)-octadecenyloxy)-prop-1-yl-N,N,N-trimethylammonium chloride (DOTMA) and 1,2-bis(oleoyloxy)-3-(trimethylammonio) propane (DOTAP).

Negatively-charged lipids include, but are not limited to, phosphatidylglycerol, phosphatidic acid, phosphatidylinositols, and phosphatidyl serines.

### V. Modes of Administration and Effective Dosages

Any method known to those in the art for contacting a cell, organ, or tissue with a sulfated polysaccharides or sulfated polysaccharide composition may be employed. Suitable methods include in vitro, ex vivo, or in vivo methods. In vivo methods typically include the administration of at least one sulfated polysaccharide, at least one liposome, at least one linked composition, or combination thereof such as those described above, to a mammal, e.g., a human. When used in vivo for therapy, the sulfated polysaccharides or sulfated polysaccharide composition is administered to the subject in effective amounts (i.e., amounts that have desired therapeutic effect). The dose and dosage regimen will depend upon the degree of the infection in the subject; the characteristics of the particular sulfated polysaccharide, liposome, or linked composition used, e.g., its therapeutic index, the subject, and the subject's history.

The effective amount is determined during pre-clinical trials and clinical trials by methods familiar to physicians and clinicians. An effective amount of a sulfated polysaccharide, liposome, linked composition, or combination thereof useful in the methods is administered to a mammal in need thereof by any of a number of well-known methods for administering pharmaceutical compounds. The sulfated polysaccharide, liposome, linked composition, or combination thereof can be administered systemically or locally.

In some implementations, the dosage for a therapeutic effect by is about 0.1 mg to about 100 mg per dose. The dosage can change depending on the active formulation, method of delivery, and individual.

In some embodiments, doses would be given about every 4 hours, 8 hours, or 12 hours, 16 hours, 24 hours, 36 hours, 48 hours, 60 hours, or 72 hours, or any ranges between any two of these values.

In some implementations, the sulfated polysaccharide, liposome, or linked composition described herein is incorporated individually or in combination into pharmaceutical compositions for administration to a subject for the treatment or prevention of viral infections described herein.

Pharmaceutical compositions are typically formulated to be compatible with its intended route of administration. Examples of routes of administration include parenteral (e.g., intravenous, intradermal, intraperitoneal or subcutaneous), oral, inhalation, iontophoretic, intranasal, and transmucosal administration.

In some implementations, the sulfated polysaccharide, liposome, or linked composition is stored in liquid, which would be delivered to the airway of humans using an inhalational delivery device.

VI. Combination Therapy with a Sulfated Polysaccharide Composition and Other Therapeutic Agents

In some implementations, the sulfated polysaccharide, liposome, linked composition, or combination thereof is combined with one or more additional agents for the prevention or treatment of viral infection. For example, current treatments for RSV include antibodies, such as palivizumab, and small molecules, such as ribavirin. In some implementations, the sulfated polysaccharide, liposome, linked composition, or combination thereof is combined with palivizumab, ribavirin, or a combination thereof.

In some implementations, the combination with another therapeutic agent produces a synergistic therapeutic effect. Therefore, lower doses of one or both of the therapeutic agents is used in treating viral infection, resulting in increased therapeutic efficacy and decreased side effects.

In some implementations, multiple therapeutic agents, e.g., liposome and ribavirin, are administered separately in any order or even simultaneously. If simultaneously, the multiple therapeutic agents can be provided in a single, unified form, or in multiple forms (by way of example only, either as a single pill or as two separate pills). In some implementations, one of the therapeutic agents is given in multiple doses. In another implementation, both therapeutic agents are given as multiple doses.

### EXAMPLES

### Example 1

### Isolation of Octasaccharide

The present example is a non-limiting implementation of a method to isolate a sulfated octasaccharide of the present technology.

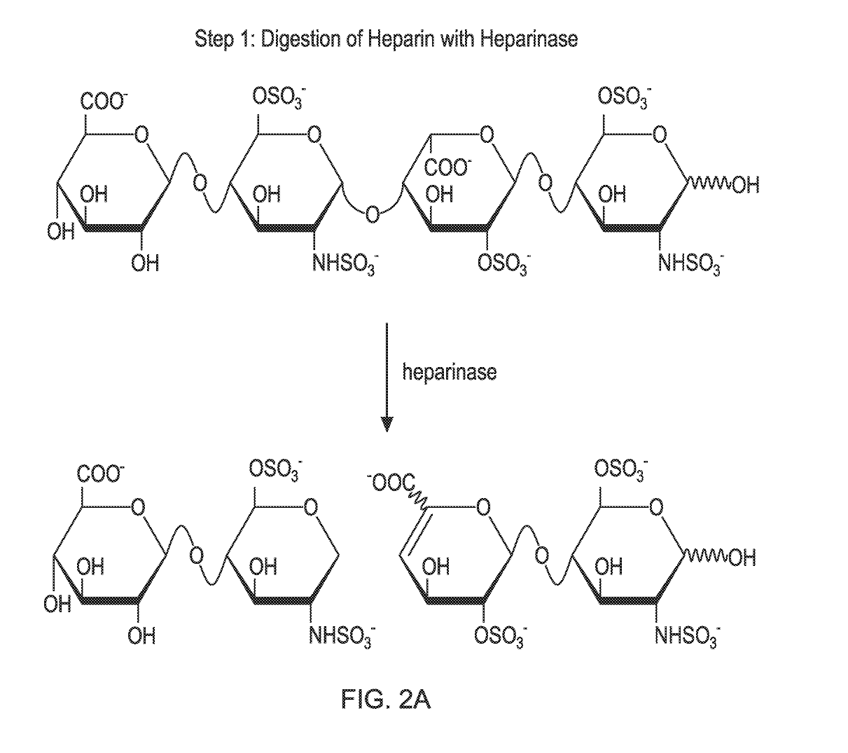

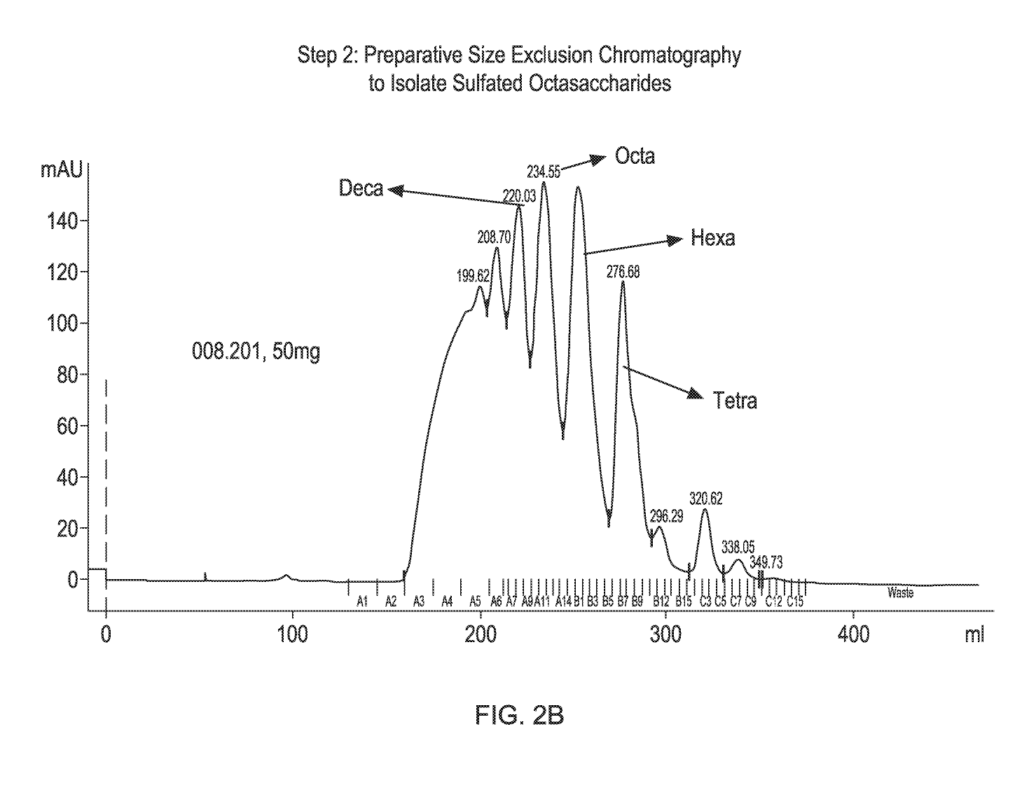

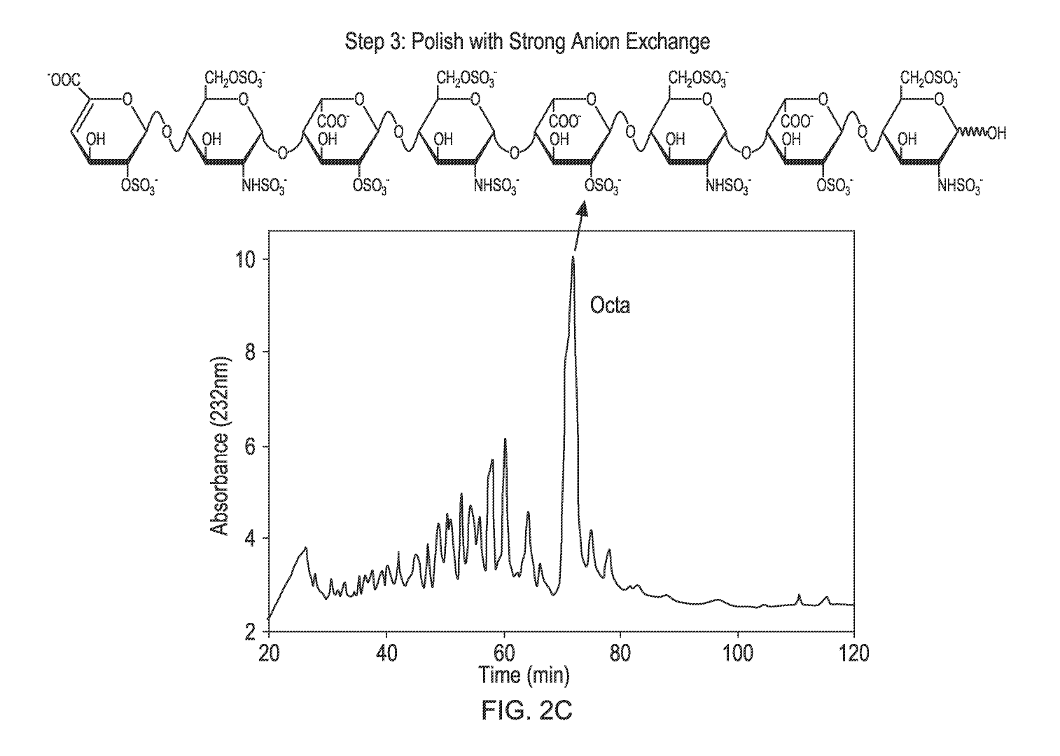

Full-length heparin (Celsus Laboratories, Cincinnati, Ohio) or heparan sulfate (bovine kidney) was subjected to digestion using chemical methods known in the art, e.g., β-elimination of a heparin benzyl ester, or by enzymatic means. FIG. 2A. 100 mg/ml of heparin was digested by heparinase I, 10 IU/mg heparin. The digestion was performed overnight at 25-30° C. in 50 mM ammonium acetate buffer, with a pH of 5.0. In some implementations, 1 mM calcium was added to the digestion.

Heparinase is a lyase, cleaving the glycosidic linkage only at selected places (yielding even-numbered saccharide chains) and leaving behind a Δ4,5 double bond, a chemical signature that can readily be monitored at 232 nm.

The mixture of digested polysaccharides was then separated into different sizes. Fast protein liquid chromatography (FPLC) was used to separate digested polysaccharides by size-exclusion chromatography. FIG. 2B. First, the digested polysaccharide mixture was frozen at −80° C., and lyophilized. The dried, digested polysaccharide mixture was then dissolved to a concentration of 10 mg/ml in either water or buffer including 5 mM Na₂HPO₄ and 150 mM NaCl at a pH of 7.2.

A 5 ml aliquot of the re-suspended digested polysaccharide mixture was injected onto a Superdex™ 30 column (GE, Pittsburg, Pa.) with a running buffer of 5 mM Na₂HPO₄ and 150 mM NaCl at a pH of 7.2. The chain length of the polysaccharides was detected at 232 nm with a UV detector. FIG. 2B. Fractions of octasaccharide were collected and buffer-exchanged using a P10 column into 10% ethanol and lyophilized to a dry powder. The exchange occurred at room temperature.

The purity of each size-separated fraction was verified using analytical GPC according to the European pharmacopeia for analysis of low molecular weight heparins.

A second purification was performed using a semi-preparative scale CarboPac® PA-200 HPLC column (9 mm dia.×250 mm length) (ThermoScientific, Bannockburn, Ill.). FIG. 2C. 2.5 mg of partially purified octasaccharide was loaded per run, and the desired separation was achieved with a 650 mM-1250 mM NaCl gradient run at 2.5 mL/minute. The sequence of the octasaccharide was determined by MALDI MS (see Rhomberg et al., Proc. Nat. Acad. Sci., 95(8):4176-81 (1998)).

### Example 2

### Glycolipid Synthesis (General)

The present example is a non-limiting implementation of a method to link sulfated polysaccharides (in this example, an octasaccharide of the present technology) to at least one lipid.

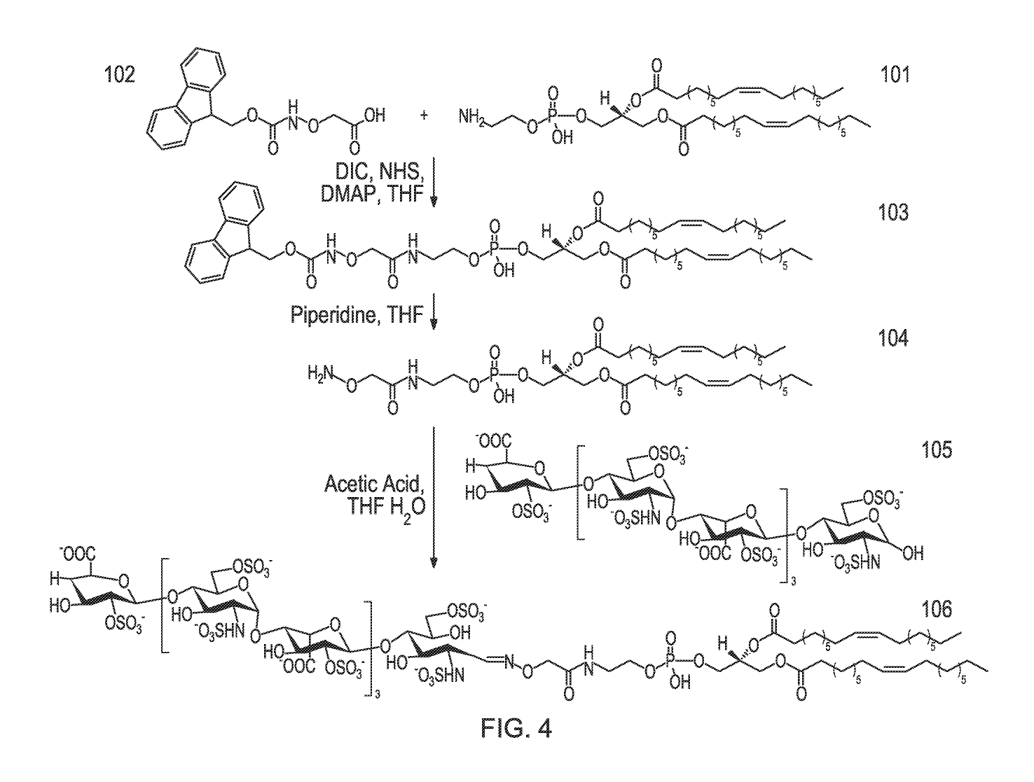

In some implementations, glycolipid synthesis is used to link a lipid to the sulfated polysaccharide. By way of example, but not by limitation, glycolipid synthesis is schematically represented in FIG. 4.

In some implementations, 1,2-dioleoyl-sn-glycero-3-phosphoethanolamine (DOPE) (Avanti Polar Lipids, Alabaster, Ala.) is the lipid attached to the polysaccharide. Referring to FIG. 4, DOPE, an amine-linked unsaturated phospholipid 101 was linked to a Fmoc-protected aminooxyacetic acid (Chem-Impex International, Wood Dale, Ill.) 102 using carbodiimide coupling. The Fmoc-modified lipid-linker conjugate 103 was purified by silica gel column chromatography from crude product. Next, piperidine (Sigma Aldrich, St. Louis, Mo.) was used to de-protect the Fmoc-modified lipid-linker conjugate 103; the reaction was monitored the reaction by thin layer chromatography (TLC). The piperidine was removed under reduce pressure, and the aminooxy-lipid-linker 104 was used in the next step without further purification. The free aminooxy group of the aminooxy-lipid-linker 104 is available for coupling to the sugar aldehyde (reducing end). 10 mg of the sulfated octasaccharide (Socta) 105 and 20 mg of the aminooxy-lipid-linker 104 were conjugated at room temperature with 3% acetic acid (Sigma Aldrich, St. Louis, Mo.) in a mixture of water and THF. The DOPE linked Socta (Socta-DOPE) 106 was purified by HPLC. The purified product was characterized by ¹H-NMR to confirm the presence of sulfated octasaccharide in the glycolipid.

### Example 3

### Method for Making Glycolipids

The present example is a non-limiting implementation of a method of preparing a glycolipid.

### Materials and Methods

The starting materials 1,2-dioleoyl-sn-glycero-3-phosphoethanolamine (DOPE) was purchased from Avanti Polar Lipids, Inc. and N-(FMoc-13-amino-4,7,10-trioxa-tridecyl)succinamic acid (linker) was purchased from Polypeptide Laboratories, San Diego. Thin layer chromatography (TLC) was performed on silica gel coated glass plates. Column chromatography was performed using silica gel 60 Å. ¹H NMR spectra were obtained using a 600 MHz Bruker instrument at 22° C.; the chemical shifts values are reported in ‘δ’ and coupling constants (J) in Hz. Mass spectrometry was performed using both 4800 MALDI-MS and MALDI-TOF (Voyager DE-STR, Applied Biosystems). Solvent evaporations were performed on a rotary evaporator under reduced pressure at 30-35° C.

Step-1: Conjugation of Lipid with Fmoc Containing Linker

Commercially available amine linked unsaturated phospholipid, DOPE, was attached with Fmoc-protected linker acids using 1-Ethyl-3-(3-dimethylaminopropyl)carbodiimide (EDC) as coupling reagents with the room temperature reaction.

1 gm of DOPE was dissolved in 10 mL of CHCl₃. 2 gm of N-(FMoc-13-amino-4,7,10-trioxa-tridecyl)succinamic acid (linker) and 2 g EDC was added and stirred for 4 hour at room temperature.

The completion of the reaction was monitored by TLC using MeOH/CHCl₃. The crude substance was extracted using dichloromethane and water. The combined organic layer was dried by rotavap to remove dichloromethane and purified by column chromatography (gravity) using silica gel and MeOH/CHCl₃ as solvent.

After purification the product solution was concentrated to remove all the organic solvent and dried by under vacuum. The purified and dried product was characterized by NMR spectroscopy and MALDI-TOF mass spectroscopy.

Step-2: Removal of Fmoc Group from the Lipid-Linker-Fmoc

The Fmoc deportation was carried out by secondary amine and formation of the amine linked product with the disappearance of Fmoc group was also monitored by TLC.

The purified product from Step 1 was dissolved in 30 mL of DMF. 10 mL of piperidine was added and stirred at room temperature for about 4-10 h. The completion of the reaction was monitored by TLC using MeOH/CHCl₃

The crude substance was extracted using dichloromethane and water. The combined organic layer was dried by rotavap to remove dichloromethane and purified by column chromatography (gravity) using silica gel and MeOH/CHCl₃ as solvent. After purification the product solution was concentrated to remove all the organic solvent and dried by under vacuum.

The purified and dried product was characterized by NMR spectroscopy and MALDI-TOF mass spectroscopy. Subsequently, the purified and characterized amine-linker-lipid conjugate was taken for the conjugation with sulfate octasaccharide via reductive beta elimination reaction.

Step 3: Combining Octasaccharide with Amine-Linker-Lipid from Step 2

The conjugation of an octasaccharide and the amine-linker-lipid conjugate was carried out at 60° C. with the presence of minimum acids.

250 mg of the amine-linker-lipid (from step 2) was dissolved in 4 ml of DMSO. 300 mg of sodium cyanoborohydride was added and the mixture was stirred at 60° C. for 5 minutes. After stirring, 500 μl of acetic acid was added, and the mixture was stirred for another 5 minutes. 10 mg sulfated octasaccharide (as produced in Example 1) was added to the mixture directly and stirred for 4 h at 60° C. After completion of the reaction the crude substance was cooled to room temperature and quenched with 100 ml of CHCl₃.

The crude mixture was purified by two step procedure. Initially the crude substance was purified by column chromatography using silica gel. After purification by column chromatography, the substance was further purified by HPLC purification.

HPLC conditions: (a) C18 column in reverse phase mode; (b) the products were injected with mixture of water and acetonitrile (7:3); (c) Solvent A: water, solvent B: acetonitrile; (d) Gradient: 20-100% of acetonitrile/water over 2 hours. The fraction in each 5 minute gap was collected and dried by speed-vac and freeze drier.

The MALDI-MS of each fraction was determined to determine the retention time of elution of the product based on the column and HPLC instrument.

In some implementations, the purified and characterized product wis further purified. The final purified product was dried by speed-vac and freeze dried and stored at −20° C.

The purified product was characterized by MALDI-MS spectroscopy to obtained the final glycolipid.

### Example 4

### Alternative Method for Making Glycolipids

The present example is a non-limiting implementation of a method of preparing a glycolipid.

The glycolipid is synthesized in 2 stages, the first stage is a large scale synthesis of a molecule containing a primary amine joined to a short, defined length, polyethyleneglycol linker, which is connected to a phospholipid (e.g., EG3-DOPE). The EG3-DOPE is used in a reductive amination reactions with reducing end sugars, which produces a stable secondary amine linkage. In the second stage a sulfated octasaccharide is then linked to the EG3-DOPE.

Synthesis of EG3-DOPE:

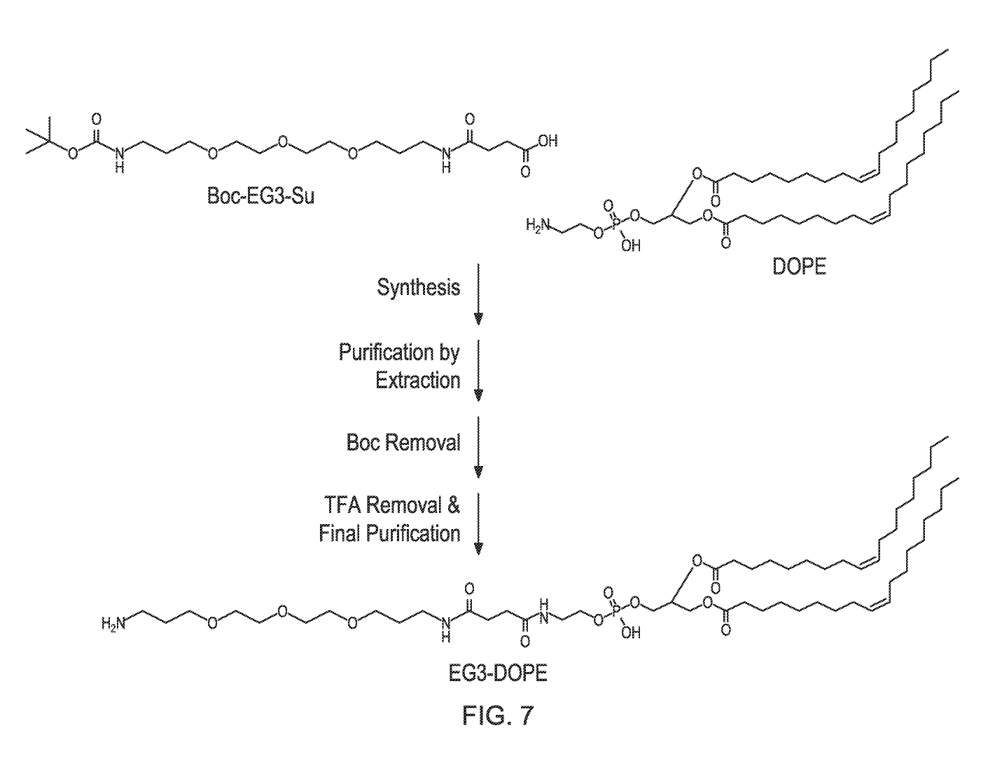

A general overview of the synthesis of EG3-DOPE is outlined in FIG. 7. 1 Eq DOPE (6.05 g; 744.03 g/mol from Corden Pharma, CAS#4004-05-1), 1.5 Eq Boc-EG3-Su (a linker) (5.13 g; 420.50 g/mol from Chemodex, CAS#250612-31-8), 2.5 Eq DMAP (2.48 g; 122.17 g/mol from Carbosynth), and 1.4 Eq EDC.HCl (2.18 g; 191.70 g/mol from Carbosynth) are combined in a 50 ml round bottom flask. The mixture is stirred overnight at 25° C. Conversion of DOPE is measured by ion trap mass spectrometry.

The above mixture is combined with dichloromethane (DCM) to a volume of 60 ml in a 250 ml separation funnel The mixture is extracted with 50 ml H₂O (about pH 9) and is back extracted with 60 ml DCM. The pooled DCM layers are extracted with 100 ml 0.1 M HCl/water, and the aqueous layer back-extracted with 60 mL DCM. The extraction with 100 ml 0.1 M HCl/water and back-extraction with 60 ml DCM is repeated 3 times, using a 500 ml separation funnel for the third extraction (about pH 1). A final extraction with 100 mL NaHCO₃ (sat.) is performed (about pH 9-10), followed by back extraction with 100 mL DCM. During the above described extraction process, organic and aqueous layers will be spotted onto UV-indicator TLC plates and UV shadowed to verify removal of UV active, water-soluble material (DMAP, EDC, EDU), and the pH of the aqueous layer was taken to verify acidity and neutralization. The DCM layers (˜300 ml) are pooled, dried with 20 g anhydrous sodium sulfate by stirring in a 600 ml beaker, filtered, rinsed with DCM and rotovapped. In some implementations, the DCM layers are stored at 4° C.

The Boc protecting group is removed by dissolving about 12 g of the DCM product above in 9 mL DCM and 4 ml MeOH, followed by addition of a mixture containing 16 ml TFA and 1 ml H₂O mix. The solution is stirred for 12 hours (with a vent needle installed via a rubber septa) at room temperature. After 12 hours, the reaction is quenched by addition of 40 ml DCM, followed by extraction with 60 mL of water, then 100 mL NaHCO₃ (sat.). The mixture is allowed to settle for 1 hour. After settling, the mixture is back extracted with 100 ml DCM. After one hour, the DCM layers are pooled and dried by adding 10% by weight anhydrous sodium sulfate and stirring the mixture for 90 minutes. The mixture is then filtered, rinsed with DCM, and split into glass scintillation vials and dried overnight in a hood. The vials are subject to high vacuum drying for about 24 hours with a 100% yield by weight (EG3-DOPE MW 1068.4, Na form). In some implementations, the vials are packaged under argon and stored at 4° C.

Synthesis of Sulfated Octasaccharide Glycolipids:

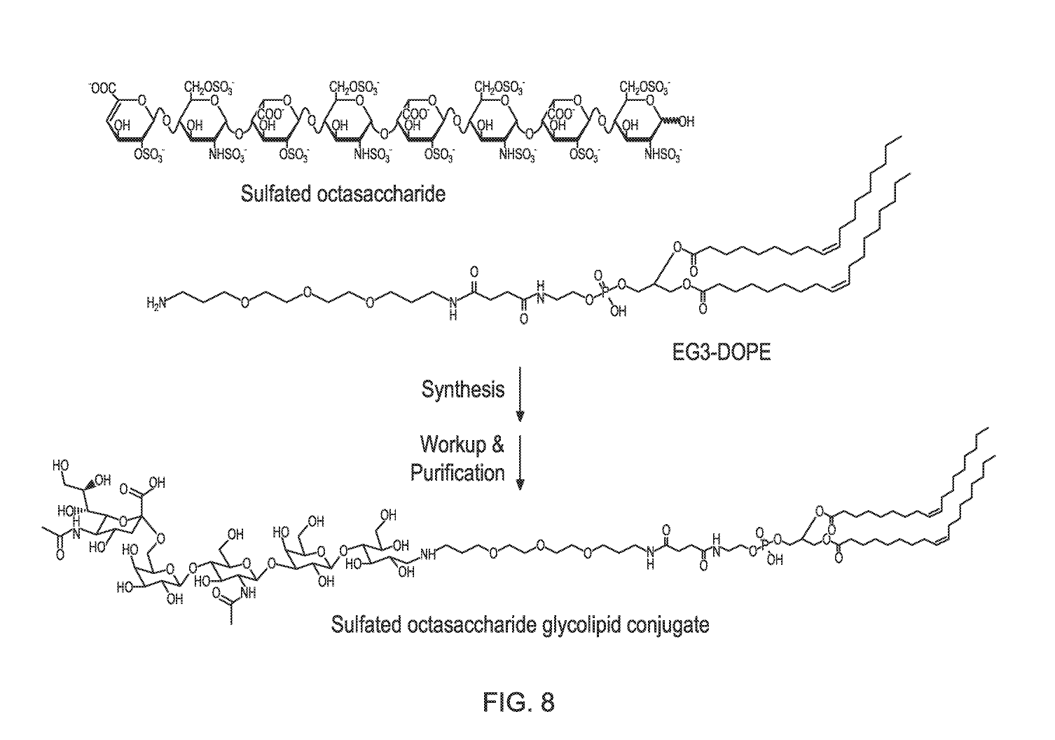

A general overview of the synthesis of sulfated octasaccharide glycolipids is outlined in FIG. 8. 0.7 ml of MeOH is added to a vial of clear, tacky EG3-DOPE (˜10 Eq 1.4×10⁻⁴ mol, 152 mg; 1046 g/mol) (described above). The vial is agitated for 20 minute to dissolve the EG3-DOPE. One equivalent of sulfated octasaccharide (as made in Example 1) is dissolved in 40-80 μl of water and is added the vial. Next 100 μl of neat HOAc (aldehyde-free from Fischer and 100 μl (˜20 Eq) of a methanolic solution of sodium cyanoborohydride (86.3 mg in 402.6 μl) added to the vial. The vial is agitated at 60° C. for 8 hours at 750 RPM in an Eppendorf Thermomixer. After 8 hours, 100 uL (˜20 Eq) sodium cyanoborohydride in methanol added to the vial and the vial is agitated at 60° C. for 8 hours at 750 RPM. The reaction course is monitored by TLC staining with p-anisaldehyde after developing in 250/175/50 DCM/MeOH/H₂O, as well as by ion trap mass spectrometry.

The glycolipid product is purified by extraction into 30 ml DCM and 20 ml NaHCO₃ (sat.). The aqueous sodium bicarbonate layer is back extracted twice with 20 ml DCM, and the final DCM pooled layers are dried with 5 g sodium sulfate (anhydrous). After filtering through a glass frit and rinsing with DCM, and rotovapping, the product is loaded onto a silica column (13×160 mm) that is prewashed in 500/240/50 DCM/MeOH/H₂O. 2 ml DCM is applied to the silica column twice, followed by adding a rinse from the original flask, and then washing the silica column twice with 2 ml of the wash solution. Fractions are eluted with 250/175/50 DCM/MeOH/H₂O, spotted onto TLC plates, briefly dried, then stained with p-anisaldehyde (13.4 ml p-anisaldehyde, 250 ml ethanol, 2.5 ml sulfuric acid) followed by heating on a hotplate to identify reaction components. Fractions are analyzed by TLC and ESI-mass spectrometry for product determination.

In some implementations, the silica columns used in the analysis of the final product is increased in length by 50%. In some implementations, the aqueous component of the elution buffer used in the analysis of the final product is decreased between about 1%-40%. In some implementations, analysis of the final product is performed using a sialic acid assay or NMR.

### Example 5

### Method for Making Liposomes

The present example is a non-limiting implementation of a making a liposome of the present technology.

Gas-tight syringes (Hamilton Co., Reno, Nev.) and 4 mL borosilicate glass vials with Teflon-lined caps (National Scientific, Rockwood, Tenn.) were thoroughly cleaned then rinsed 10× with 100% ethanol and then 10× with chloroform. Vials were soaked in 300 mM HCl for 1.5 h and then rinsed thoroughly with water, 3× with ethanol, and 3× with chloroform. Residual solvent was evaporated under a filtered stream of dry nitrogen gas. Glycolipids, e.g., made in Example 2, and a mixture of lipids were mixed and deposited in the clean vials using clean syringes. In some implementations, the final mixture is 7.5 mol % glycolipid, 30 mol % cholesterol, and 62.5 mol % DOPC. In another implementation, the final mixture is 30 mol % glycolipid, 30 mol % cholesterol, and 39 mol % DOPC. In some implementations, the final mixture contains cholesterol at about 1 mol %, about 3 mol %, about 6 mol %, about 9 mol %, about 12 mol %, about 15 mol %, about 18 mol %, about 21 mol %, about 24 mol %, about 27 mol %, about 30 mol %, or ranges between any two of these values. In some implementations, the final mixture contains glycolipids at about 5 mol %, about 10 mol %, about 15 mol %, about 20 mol %, about 25 mol %, about 30 mol %, about 35 mol %, about 40 mol %, about 45 mol %, about 50 mol %, or ranges between any two of these values. In some implementations, the solvent for cholesterol and phospholipid is chloroform. In some implementations, the solvent for the glycolipid is water. In some implementations, the above mixtures were mixed at room temperature.

Solvent was evaporated under a filtered stream of dry nitrogen gas while manually rotating the vial until only a thin layer of a lipid film remained on the inner walls. Residual solvent was removed by placing uncapped vials in a dessicator (Dry Seal, Wheaton, Millville, N.J.), then followed by application of reduced pressure for 24 hours using an oil-free diaphragm vacuum pump (Gast, Benton Harbor, Mich.).

Aqueous lipid solutions were made by hydrating the lipid film in 150 mM phosphate buffered saline (PBS) (140 mM NaCl, 8.5 mM NaH₂PO₄, 1.5 mM Na₂HPO₄, pH 7.4) and vortexing for 2 min in 30 second intervals. The lipid solution was then subjected to 10 rapid cycles of freeze-thawing by submersion in liquid nitrogen and 70° C. water, respectively, to break apart multilamellar structures.

The lipid solution was extruded through 200 nm pores. In some implementations, extrusion consisted of 10 passes through an aluminum oxide membrane using a Lipex™ Thermobarrel Extruder (Northern Lipids, Burnaby, BC, Canada). In an alternative implementation, extrusion consisted of 21 passes through a polycarbonate membrane using a LiposoFast-Basic Extruder (Avestin; Ottawa, ON, Canada). Before extrusion, the Extruders were cleaned and primed with 150 mM phosphate buffered saline (8.5 mM Na₂HPO₄, 1.5 mM NaH₂PO₄, and 140 mM NaCl) at pH 7.6. Priming the LiposoFast-Basic Extruder consisted of rising all parts with the phosphate buffer solution and passing 500 μl of phosphate buffer through the extruder 21 times. Priming the Lipex™ Thermobarrel Extruder consisted of rising all parts with the water and passing 5 ml of phosphate buffer through the extruder 5 times.

After the final pass, samples were collected in a clean vial, sealed with a Teflon-lined cap, and stored at 4° C. until use. Lipid concentration post-extrusion relative to pre-extrusion was determined by fluorimetry. Typical recoveries were ˜50% with the Lipex™ Thermobarrel Extruder and ˜80% with the LiposoFast-Basic Extruder.

Diameter and polydispersity of the liposomes were determined by dynamic light scattering (Zetasizer Nano; Malvern Instruments, Worcestershire, UK) specifying a lipid refractive index of 1.480 and a dispersant (when PBS) refractive index of 1.332. Measurements were taken using 40 μL disposable cuvettes at room temperature (20° C.) and a backscattering angle of 173 degrees. Average liposomes were 90 to 150 nm in diameter with a polydispersity of 0.1 to 0.2.

### Example 6

### Method for Blocking Respiratory Syncytial Virus Infection In Vitro Using Sulfated Octasaccharides

The ability of purified sulfated octasaccharide, in solution, to block respiratory syncytial virus (RSV) infection of Vero cells was examined. The blocking ability of purified sulfated octasaccharide was compared to heparin sodium.

### Method and Materials

Purified sulfated octasaccharide was prepared according to the method of Example 1. Vero cells were seeded into 24-well plates and incubated at 37° C. for 24 hours to form monolayers. Samples were diluted to the desired concentration in sterile DMEM in a final volume of 225 μl. Full-length heparin sodium (the starting material) or purified HSocta was added to Vero cells simultaneously with RSV that was diluted to 300 PFU/mL then incubated at 37° C. for 30 min. Vero cells were washed with PBS and samples were incubated at 37° C. for 1 hour on Vero cells and then washed with PBS and incubated with DMEM containing 10% FBS and incubated at 37° C. for 72 h. Cells were fixed and stained with anti-glycoprotein F and anti-glycoprotein G antibodies (MAB858-2 and MAB8262F Millipore, Billerica, Mass.). Plaques were visualized with anti-mouse horseradish peroxidase-conjugated secondary antibody (BD Biosciences, San Jose, Calif.) and developed with peroxidase substrate kit (Vector Laboratories, Burlingame, Calif.). Viral plaques in the Vero monolayer were counted and the PFU/mL was determined.

### Results

The purified heparan sulfate octasaccharide (HSocta) had strong anti-RSV properties when tested for its ability to block infection of MDCK cells. Each material showed a significant ability to block RSV infection, with heparin sodium inhibiting >90% of PFU formation at a concentration of 1 μg/ml indicating that the native heparin displays surface glycans that very effectively bind RSV. The purified octasaccharide had less activity, blocking PFU formation roughly 50% at 1 μg/ml or higher.

The results show that purified sulfated octasaccharides of the present technology are useful in inhibiting the infectivity of influenza virus. The results show that the compositions of the present technology are useful in the treatment or prevention of viral infection.

### Example 7

### Method for Blocking RSV Infection In Vitro Using Sulfated Octasaccharide Liposomes

In some implementations, displaying purified octasaccharide on the surface of a liposome provides for multivalent binding to the virus, while eliminating the anticoagulant and other detrimental effects of native heparin.

The ability of liposomes to block RSV infection of Vero cells was examined. The blocking ability of liposomes was compared to purified sulfated octasaccharide, in solution, and a control, i.e., a liposome without sulfated octasaccharides.

### Method and Materials

Liposomes were prepared using the method described in Example 3. Briefly, a lipid mixture containing 7.5% isolated, purified sulfated octasaccharide linked to DOPE, 30% cholesterol, and 62.5% DOPC was extruded through a 200 nm membrane to produce liposomes that had a 140 nm diameter.

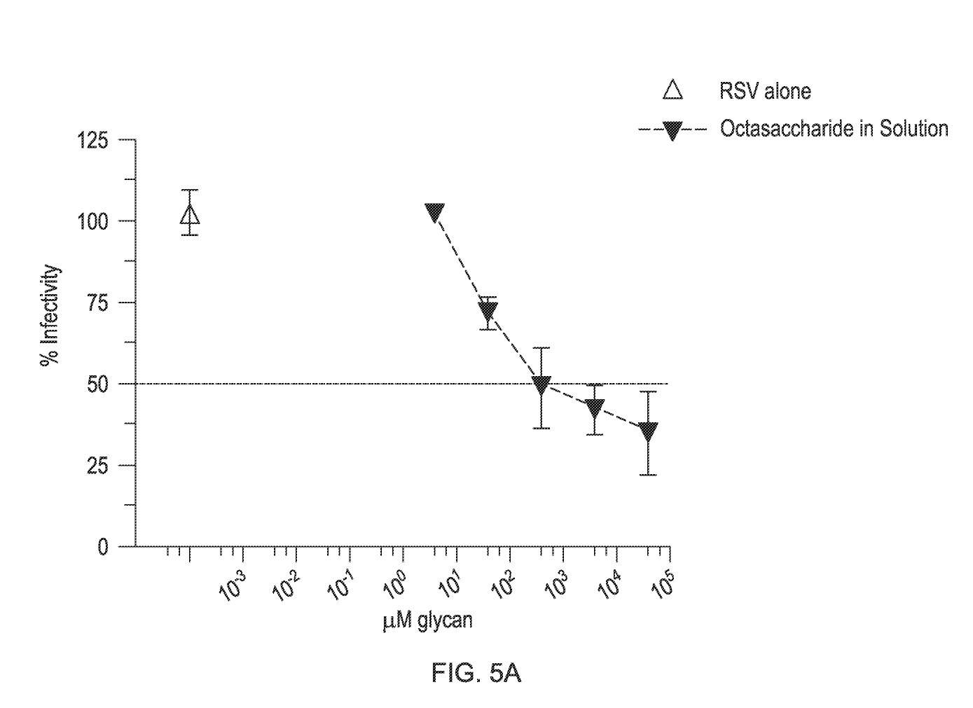

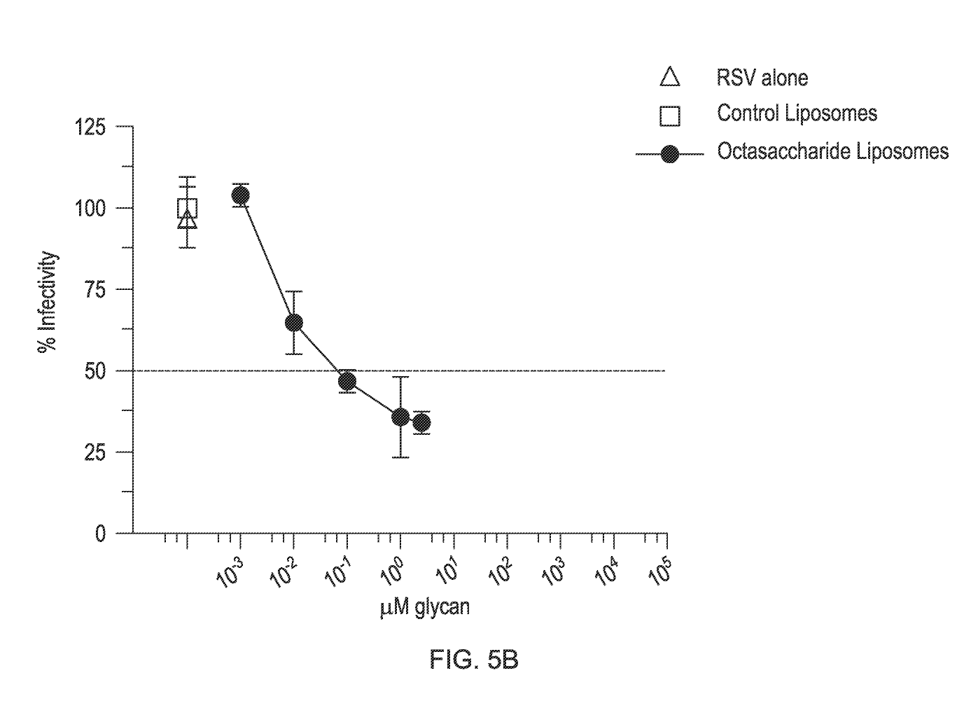

Purified sulfated octasaccharide as described in Example 1, in solution or incorporated into liposomes, at the concentrations indicated were assessed using a plaque assay. Confluent Vero cell monolayers were formed by adding 1×10⁶ Vero cells to wells of a 12-well plate then incubating the cells in 1 ml Dulbecco's Modified Eagle Medium (DMEM) with 4 mM glutamine and 10% fetal bovine serum as growth medium. The medium was removed from the cells. Sulfated octasaccharide, liposomes, or buffer at the concentrations indicated (see FIG. 5) were mixed with 200 PFU RSV strain A2 in 150 mM phosphate buffered saline for 30 min at 37° C. and the mixture was added to the monolayer surface. After one hour, the mixtures were removed, and the cells were washed and overlaid with 1 ml plaquing medium (DMEM with 4 mM glutamine, 2% FBS, and 0.4% agarose). After 5 to 7 days incubation at 37° C., the number of RSV plaques was counted. Liposome treated virus was compared to untreated virus, which represented 100% infectivity.

### Results

The liposome was over 1000× more effective at inhibiting RSV infection of Vero cells as compared to purified sulfated octasaccharide, in solution. FIG. 5. A 50% reduction in RSV infectivity was achieved with roughly 500 μM of purified sulfated octasaccharide, in solution, compared to 100 nM of sulfated octasaccharide composition, FIG. 5. Control liposomes lacking sulfated octasaccharide had no effect on RSV infectivity.

The results show that sulfated octasaccharides and liposome of the present technology are useful in blocking the infectivity of the RSV. The results also indicated that the multivalent structure of the liposome greatly enhances the ability of the sulfated octasaccharide to inhibit infection by RSV. The results show that the compositions of the present technology are useful in the treatment or prevention of viral infection.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

What is claimed is:

1. A sulfated polysaccharide comprising GlcUA(2S)-GlcNS(6S)-Ido(2S)-GlcNS(6S)-Ido(2S)-GlcNS(6S)-Ido(2S)-GlcNS(6S).

2. A polysaccharide composition comprising at least one glycolipid, wherein the glycolipid comprises at least one sulfated polysaccharide linked to a lipid and wherein the sulfated polysaccharide comprises between 3 to 10 monosaccharide units.

3. The composition of claim 2, further comprising a mixture of lipids, wherein the mixture of lipids and at least one glycolipid are in the form of a liposome, wherein the sulfated polysaccharide is displayed on a surface of the liposome.

4. The composition of claim 2, wherein the sulfated polysaccharide comprises between 1-4 sulfur atoms per disaccharide.

5. The composition of claim 2, wherein the sulfated polysaccharide comprises an octasaccharide, wherein the octasaccharide comprises three sulfur atoms per disaccharide.

6. The composition of any one of claim 4 or 5, wherein the sulfur atoms are in the form of a sulfate.

7. The composition of claim 2, wherein the sulfated polysaccharide comprises at least one monosaccharide select from the group consisting of Ido(2S), Iduronate, 2-O sulfo-iduronate, and 2,3-split uronic acid.

8. The composition of claim 2, wherein the sulfated polysaccharide is GlcUA(2S)-GlcNS(6S)-Ido(2S)-GlcNS(6S)-Ido(2S)-GlcNS(6S)-Ido(2S)-GlcNS(6S).

9. The composition of claim 2, wherein the composition inhibits a viral infection.

10. The composition of claim 9, wherein the viral infection is selected from the group consisting of respiratory syncytial virus (RSV), influenza virus, herpes simplex viruses 1 and 2, cytomegalovirus (CMV), vaccinia virus, vesicular stomatitis virus (VSV), Sindbis virus, HIV, human papillomavirus (HPV), influenza A virus, and alphaviruses.

11. The composition of claim 9, wherein the composition binds to the F and G envelop glycoproteins.

12. A method of treating or preventing a viral infection comprising administering a therapeutic amount of a polysaccharide composition, the composition comprising at least one glycolipid, wherein the glycolipid comprises at least one sulfated polysaccharide linked to a lipid and wherein the sulfated polysaccharide comprises between 3 to 10 monosaccharide units.

13. The method of claim 12, wherein the polysaccharide composition further comprises a mixture of lipids, wherein the mixture of lipids and at least one glycolipid are in the form of a liposome, wherein the sulfated polysaccharide is displayed on a surface of the liposome.

14. The method of claim 12, wherein the sulfated polysaccharide composition further comprises a carrier molecule or support substrate, wherein at least one glycolipid is linked to the surface of the carrier molecule or the support substrate.

15. The method of claim 14, wherein the carrier molecule or the support substrate is selected from the group consisting of peptides, polypeptide, protein, carbohydrate, nanoparticles, polymers, glass beads, magnetic particles, nucleic acid, small molecule, cells, virus, dendrimer, particle, bead, macromolecule, and solid lipid particle.

16. The method of claim 12, wherein the sulfated polysaccharide comprises between 1-4 sulfur atoms per disaccharide.

17. The method of claim 12, wherein the sulfated polysaccharide comprises an octasaccharide, wherein the octasaccharide comprises three sulfur atoms per disaccharide.

18. The method of claim 16, wherein the sulfur atoms are in the form of a sulfate.

19. The method of claim 12, wherein the sulfated polysaccharide comprises at least one monosaccharide select from the group consisting of Ido(2S), Iduronate, 2-O sulfo-iduronate, and 2,3-split uronic acid.

20. The method of claim 12, wherein the sulfated polysaccharide is GlcUA(2S)-GlcNS(6S)-Ido(2S)-GlcNS(6S)-Ido(2S)-GlcNS(6S)-Ido(2S)-GlcNS(6S).

21. The method of claim 12, wherein the viral infection is caused by one or more viruses selected from the group consisting of respiratory syncytial virus (RSV), influenza virus, herpes simplex viruses 1 and 2, cytomegalovirus (CMV), vaccinia virus, vesicular stomatitis virus (VSV), Sindbis virus, HIV, human papillomavirus (HPV), influenza A virus, and alphaviruses.
